SEQUENCE LISTING (16 sequences)

SEQ ID NO: 1 - protein, 229 aa, Artificial Sequence
  Variable light chain and heavy chain sequence of antibody fragment binding to EpCAM
HDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKREVQLVQSGPGLVQPGGSVRISCAASGYTFTNYGMNWVKQAPGKGLEWMGWINTYTGESTYADSFKGRFTFSLDTSASAAYLQINSLRAEDTAVYYCARAIKGDYWGQGTLLTVSS

SEQ ID NO: 2 - protein, 669 aa, Artificial Sequence
  Sequence of immunoconjugate VB4-845
MKYLLPTAAAGLLLLAAQPAMAHHHHHHDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRATPSHNSHQVPSAGGPTANSGTSGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSEFGGAPEFPKPSTPPGSSGLEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPHHHHHHKDEL

SEQ ID NO: 3 - DNA, 2084 bp, Artificial Sequence
  Nucleic acid sequence of VB4-845
gaattcctgcaggtctatggaacgataaatgcccatgaaaattctatttcaaggagacagtcataatgaaatacctattgcctacggcagccgctggattgttattactcgctgcccaaccagcgatggcgcaccatcatcaccatcacgatatccagatgacccagtccccgtcctccctgagtgcttctgttggtgaccgtgttaccatcacctgccgttccaccaaatccctcctgcactccaacggtatcacctacctttattggtatcaacagaaaccgggtaaagctccgaaacttctgatctaccagatgtccaacctggcttccggtgttccgtctcgtttctccagttctggttctggtaccgacttcaccctgaccatctcttctctgcagccggaagacttcgctacctactactgcgctcagaacctggaaatcccgcgtaccttcggtcagggtaccaaagttgaacttaagcgcgctaccccgtctcacaactcccaccaggttccatccgcaggcggtccgactgctaactctggaactagtggatccgaagtacagctggttcagtccggcccgggtcttgttcaaccgggtggttccgttcgtatctcttgcgctgcttctggttacacgttcaccaactacggcatgaactgggtcaaacaggctccgggtaaaggcctggaatggatgggctggatcaacacctacaccggtgaatccacctacgctgactccttcaaaggtcgcttcactttctccctcgacacaagtgctagtgctgcatacctccaaatcaactcgctgcgtgcagaggatacagcagtctattactgcgcccgtttcgctatcaaaggtgactactggggtcaaggcacgctgctgaccgtttcctcggaatttggtggcgcgccggagttcccgaaaccgtccaccccgccgggttcttctggtttagagggcggcagcctggccgcgctgaccgcgcaccaggcctgccacctgccgctggagactttcacccgtcatcgccagccgcgcggctgggaacaactggagcagtgcggctatccggtgcagcggctggtcgccctctacctggcggcgcgactgtcatggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccagcgccgacgtggtgagcctgacctgcccggtcgccgccggtgaatgcgcgggcccggcggacagcggcgacgccctgctggagcgcaactatcccactggcgcggagttcctcggcgacggtggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggatctcgacgcgatctggcgcggtttctatatcgccggcgatccggcgctggcctacggctacgcccaggaccaggaacccgacgcgcgcggccggatccgcaacggtgccctgctgcgggtctatgtgccgcgctccagcctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggtggcgacctcgacccgtccagcatccccgacaaggaacaggcgatcagcgccctgccggactacgccagccagcccggcaaaccgccgcatcaccaccatcaccataaagacgaactgtagtgactcgag

SEQ ID NO: 4 - protein, 16 aa, Artificial Sequence
  Light chain CDR1
RSTKSLLHSNGITYLY

SEQ ID NO: 5 - protein, 7 aa, Artificial Sequence
  Light chain CDR2
QMSNLAS

SEQ ID NO: 6 - protein, 9 aa, Artificial Sequence
  Light chain CDR3
AQNLEIPRT

SEQ ID NO: 7 - protein, 5 aa, Artificial Sequence
  Heavy chain CDR1
NYGMN

SEQ ID NO: 8 - protein, 16 aa, Artificial Sequence
  Heavy chain CDR2
WINTYTGESTYADSFK

SEQ ID NO: 9 - protein, 7 aa, Artificial Sequence
  Heavy chain CDR3
FAIKGDY

SEQ ID NO: 10 - DNA, 64 bp, Artificial Sequence
  Tox1 DNA primer
ctcggaattcggtggcgcgccggagttcccgaaaccgtccaccccgccgggttcttctggttta

SEQ ID NO: 11 - DNA, 60 bp, Artificial Sequence
  Tox2 DNA primer
gtcaagcttctacagttcgtctttatggtgatggtggtgatgcggcggtttcccgggctg

SEQ ID NO: 12 - DNA, 73 bp, Artificial Sequence
  DNA primer
ctagataacgagggcaaaaaatgaaaaagacagctatcgcgattgcagtggcactggctggtttcgctaccgt

SEQ ID NO: 13 - DNA, 48 bp, Artificial Sequence
  DNA primer
gccactgcaatcgcgatagctgtctttttcattttttgccctcgttat

SEQ ID NO: 14 - DNA, 34 bp, Artificial Sequence
  DNA primer
agcgcaggccgaccaccatcatcaccatcacgat

SEQ ID NO: 15 - DNA, 55 bp, Artificial Sequence
  DNA primer
atcgtgatggtgatgatggtggtcggcctgcgctacggtagcgaaaccagccagt

SEQ ID NO: 16 - DNA, 2084 bp, Artificial Sequence
  Codon optimized sequence of VB4-845
gaattcctgcaggtctatggaacgataaatgcccatgaaaattctatttcaaggagacagtcataatgaaatatctgctgccgactgctgctgcgggtctgctgctgctcgcggctcagcctgctatggcacaccaccatcaccaccatgacatccagatgacccagtctcctagctctcttagcgcaagcgtaggtgaccgtgtgaccatcacctgccgtagcactaaatctctgctgcatagcaacggcatcacctacctgtattggtaccagcagaaaccgggtaaagctccgaaactgctgatctaccagatgtctaacctggctagcggtgttcctagccgttttagctctagcggtagcggtactgacttcaccctgaccattagctctctgcagcctgaagacttcgcgacctactactgcgcccagaaccttgaaatcccgcgtactttcggccagggtaccaaagtcgaactgaaacgtgcgaccccgagccataactctcaccaggttcctagcgcaggtggtccgactgctaactctggcactagcggttctgaagttcagctcgttcagtctggtccgggtctggttcagccgggtggtagcgttcgtatctcttgcgcggcttctggttacaccttcactaactacggtatgaactgggttaaacaggctccgggtaaaggtctggagtggatgggttggattaacacctacacgggtgaatctacctacgccgatagcttcaaaggtcgtttcacctttagccttgacacctctgcgtctgcggcgtaccttcagatcaactctctgcgtgctgaggacaccgctgtttactactgcgctcgtttcgcgattaaaggtgactattggggccagggcaccctgctgaccgttagctctgagttcggtggtgcgcctgagttccctaaaccgtctaccccgccaggttcttctggtcttgaaggtggtagcttggcagcgttgaccgcacaccaagcatgccacctgccgctggagaccttcacccgtcaccgtcagccgcgtggttgggaacagctggagcagtgcggttatccggttcagcgtctggtagctctgtacctggctgctcgtctgagctggaaccaggttgaccaggtgatccgtaacgcgctcgctagcccgggttctggtggtgacctgggtgaagctatccgtgaacagccggaacaagctcgtctcgcgttgacccttgctgcagcggaatctgaacgtttcgttcgtcagggtaccggtaacgatgaagctggtgcagcgtctgcggatgtagttagcctgacttgcccggttgcggctggtgaatgcgctggtccggcagactctggtgacgcgttgcttgaacgtaactacccgaccggtgctgagttcctgggtgacggtggtgacgttagctttagcacccgtggtacccagaactggaccgttgaacgtctgctgcaggctcaccgtcagcttgaagaacgtggttatgttttcgtaggttaccacggtaccttcctggaggcggcgcagtctatcgtgttcggtggtgttcgtgctcgttcccaggaccttgacgcaatttggcgcggtttctacatcgcgggtgacccagcgctcgcgtacggttacgctcaggaccaggaaccggatgcgcgtggtcgtattcgtaacggtgcactgctgcgtgtgtacgttcctcgttctagcctgccgggtttctatcgtaccggtctgaccctggctgccccggaagccgcgggtgaagttgaacgtctgatcggtcaccctctgccgctgcgtctggatgcgatcaccggtccggaagaagaaggtggtcgtcttgaaaccatcctgggttggccgttggctgagcgtactgtagtcatcccgtctgcgatcccgaccgacccgcgtaacgtaggtggtgaccttgacccgtctagcatcccggataaagaacaggccatcagcgcactgccggactacgcgtctcaaccgggtaaaccgccgcaccaccatcatcaccacaaagatgaactgtagtgactcgag

(19) United States
(12) Patent Application Publication
(10) Pub. No.: US 2016/0060352 A1
(43) Pub. Date: Mar. 3, 2016

(54) COMPOSITIONS AND METHODS FOR DETECTION AND TREATMENT OF HEPATOCELLULAR CARCINOMA

(71) Applicant: VIVENTIA BIO INC., Winnipeg (CA)

(72) Inventors: Shinji Tanaka, Tokyo (JP); Glen MacDonald, Winnipeg (CA)

(21) Appl. No.: 14/783,994

(22) PCT Filed: Apr. 14, 2014
(86) PCT No.: PCT/CA2014/050373

§ 371 Date: Oct. 12, 2015

Related U.S. Application Data
(60) Provisional application No. 61/811,360, filed on Apr. 12, 2013.

(51) Int. Cl. C07K 16/30; A61K 38/16; C07K 14/21; A61K 39/395; G01N 33/574; A61K 31/513

(52) U.S. Cl. CPC C07K 16/303; G01N 33/57438; A61K 31/513; C07K 14/21; A61K 39/39558; A61K 38/164; G01N 2333/705; C07K 2317/622; C07K 2319/55; A61K 2039/505

(57) ABSTRACT

Disclosed herein are compositions and methods to treat hepatocellular carcinoma. In one embodiment, a method of treating a subject with hepatocellular carcinoma comprises administering a therapeutically effective amount of an immunoconjugate (VB4-845) comprising an antibody conjugated to an effector molecule, and wherein the antibody recognizes epithelial cell adhesion molecule (Ep-CAM). The effector molecule may be Pseudomonas exotoxin A. In some embodiments, the immunoconjugate may be co-administered, concurrently administered, or sequentially administered with one or more other anticancer agents.

### CROSS-REFERENCE TO RELATED APPLICATIONS

This application claims priority to U.S. Provisional Application No. 61/811,360 filed on Apr. 12, 2013, which is hereby incorporated by reference in its entirety.

### GOVERNMENT INTERESTS

Not Applicable

### BRIEF SUMMARY OF THE INVENTION

The present disclosure is related to compositions and methods for treating hepatocellular carcinomas. In one embodiment, a method of treating a subject with hepatocellular carcinoma comprises administering to said subject a therapeutically effective amount of an immunoconjugate comprising an antibody conjugated to an effector molecule, wherein the antibody recognizes epithelial cell adhesion molecule (Ep-CAM). In some embodiments, the antibody comprises light chain complementarity determining regions comprising the amino acid sequences defined by SEQ ID NOS: 4, 5 and 6, and heavy chain complementarity determining regions comprising the amino acid sequences defined by SEQ ID NOS: 7, 8, and 9. In some embodiments, an effector molecule may be a toxin such as abrin, modeccin, viscumin, gelonin, bouganin, saporin, ricin, ricin A chain, bryodin, luffin, momordin, restrictocin, Pseudomonas exotoxin A, pertussis toxin, tetanus toxin, botulinum toxin, Shigella toxin, cholera toxin, diphtheria toxin and the like. In some embodiments, the immunoconjugate is administered directly to the cancer site.

In an additional embodiment, the immunoconjugate is VB4-845 as shown in SEQ ID NO. 2, or a variant thereof. In some embodiments, the immunoconjugate may be co-administered, concurrently administered, or sequentially administered with one or more other anticancer agents.

In another embodiment, a method of detecting or monitoring a carcinoma in a subject is disclosed. For example, the detection of hepatocellular carcinoma may include: contacting a test sample taken from said subject with an antibody to form an antibody-antigen complex, wherein the antibody comprises light chain complementarity determining regions comprising the amino acid sequences defined by SEQ ID NOS: 4, 5 and 6, and heavy chain complementarity determining regions comprising the amino acid sequences defined by SEQ ID NOS: 7, 8, and 9; measuring the antibody-antigen complex in the test sample; and normalizing the results against a control.

In a further embodiment, a kit for diagnosing carcinoma is disclosed. For example, a kit for diagnosing hepatocellular carcinoma comprises an antigen comprising light chain complementarity determining regions comprising the amino acid sequences defined by SEQ ID NOS: 4, 5 and 6, and heavy chain complementarity determining regions comprising the amino acid sequences defined by SEQ ID NOS: 7, 8, and 9; and instructions for the use thereof.

In an additional embodiment, a method of killing liver cancer cells in vitro or in vivo involves contacting the liver cancer cells to an effective amount of an immunoconjugate comprising an antibody conjugated to an effector molecule, and wherein the antibody recognizes epithelial cell adhesion molecule (Ep-CAM).

### DESCRIPTION OF DRAWINGS

FIG. 1. Map of VB4-845. The map depicts the organization of the immunoconjugate's linked 4D5MOCB scFv and ETA₂₅₂₋₆₀₈ portions, as well as the various domains, including the histidine tags, PelB signal, linker regions, the V_{L} and V_{H} regions, ETA regions II, Ib, and III, and the ER retention signal.

FIG. 2 shows the SEQ ID NOS: 2 and 3 that correspond to amino acid and nucleic acid sequences of VB4-845, with pelB leader sequence. The nucleotide and polypeptide sequences can be divided into domains including: the signal sequence for periplasmic expression, histidine tags, CDR 1, 2 and 3 domains, V_{L} domain, V_{H} domain, linkers, ETA domains II, Ib, III, and an ER retention signal KDEL.

FIG. 3 shows immunohistochemical staining and histograms of patient prognosis. (A) Immunohistochemical analysis of Ep-CAM expression in CM-type HCC cases. (a) a typical CM-type HCC showing Ep-CAM expression. (b) a typical CM-type HCC showing no Ep-CAM expression. Membranous staining of Ep-CAM in the cancer cells and bile ducts, but not in the adjacent noncancerous cells (magnification, ×100). BD, bile duct. Postoperative prognosis of patients with CM-type HCC with (+) or without (−) expression of Ep-CAM protein. (B), overall survival curves and (C), recurrence-free survival curves after curative operation. In the patients with CM-type HCC, Ep-CAM expression was significantly associated with the poor prognosis after curative operation. Log-rank test demonstrated statistically significant differences in overall and recurrence-free survival rates (p=0.0447 and p=0.0171, respectively).

FIG. 4 demonstrates association of expression of Ep-CAM with in vitro effects of VB4-845 and 5-FU in human HCC cell lines. (A) Expression of Ep-CAM was analyzed by flow cytometry in 8 HCC cell lines. (B and C) Inhibition of tumor cell growth upon treatment with VB4-845 or 5-FU is shown. Eight HCC cell lines were incubated for 72 h with VB4-845 at concentrations ranging from 0.01 to 10 pM or 48 h with 5-FU at concentrations ranging from 0.01 to 100 μg/ml. Cell growth was measured in MTS assays. The graph shows the mean values; the error bars shows standard deviations from three determinations. (D) Cell proliferation assay of 8 HCC cell lines with VB4-845 (1 pM for HepG2 and Hep3B and 10 pM for the remaining cell lines) and 5-FU (5 μg/ml for HLE, HLF, and PLC/PRF/5 cells and 1 μg/ml for the remaining cells) for 48 h. Columns, alive cells (%); vertical bars, standard deviation.

FIG. 5 shows sphere formation in Ep-CAM^{high} cell lines after the treatment of VB4-845, 5-FU, and the combination of VB4-845 plus 5-FU using 3D culture system after 7 days of culture (200×). Control cells and the surviving cells after the treatment of 5-FU formed spheres but the surviving cells after the treatment of VB4-845 and the combination of VB4-845 plus 5-FU did not form spheres. Scale bar, 50 μm.

FIG. 6 shows FACS analysis of Ep-CAM^{high} cell lines based on various stem/progenitor markers after the treatment of VB4-845, 5-FU, and the combination of VB4-845 plus 5-FU. (A) A representative result of three independent staining experiments is shown and the positive rate of markers corresponding to the graph is indicated. Arrow shows a unique bimodal pattern of HepG2 cells for CD133 expression. (B and C) The expression of CD133 after the treatment of VB4-845 or 5-FU is shown. Columns, alive cells (%); vertical bars, standard deviation. (D and E) The expression of CD13 after the treatment of VB4-845 or 5-FU is shown. Columns, alive cells (%); vertical bars, standard deviation.

FIG. 7 shows results of in vivo studies in subcutaneous xenograft models. Established subcutaneous xenografts of HuH-7 were treated with intravenous injection of control saline or VB4-845 30 μg/kg and intraperitoneal injection of control saline or 5-FU 30 mg/kg three times per week for 2 weeks. (A) Representative subcutaneous tumors in mice at the end of the dosing period are shown. Scale bar, 10 mm. (B) Tumor volumes plotted every other day in the four groups (n=10) are shown. Arrows indicate the time of administration. Vertical bars, standard error. Statistical analysis was done by two-tailed Student's t test (p<0.05).

FIG. 8 illustrates in vivo studies in liver orthotopic xenograft models. Established liver orthotopic xenografts of HuH-7 were treated with control saline or the combination of VB4-845 plus 5-FU. The method and schedule of administration was the same as the subcutaneous tumor. (A) Representative liver tumor in mice at the end of the dosing period is shown. Scale bar, 10 mm. (B) Liver tumor volume analyzed 2 weeks after administration of the control (1964±367 mm3) or the combination of VB4-845 plus 5-FU (141±34 mm3) (n=5) is shown. Vertical bars, standard error. Statistical analysis was done by two-tailed Student's t test (p=0.0011). (C) H&E staining and immunostaining of Ep-CAM (magnification, ×40) is shown. (D) The percentage of strongly stained tumor cells in all of tumor cells between two groups is shown. Vertical bars, standard deviation.

FIG. 9 shows concentration dependent reduction in mammosphere forming efficiency (MFE) by VB4-845 when tested at different concentrations. Vertical bars, standard deviation.

FIG. 10 displays re-plating assay where cells previously exposed to VB4-845 were washed and re-plated in mammosphere growth media. Staining with trypan blue indicated VB4-845 was cytotoxic, but not cytostatic, since dye was excluded from viable cells (A) but not from dead cells (B).

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Nothing herein is to be construed as an admission that the invention is not entitled to antedate such disclosure by virtue of prior invention.

As used herein and in the appended claims, the singular forms “a”, “an”, and “the” include plural reference unless the context clearly dictates otherwise. Thus, for example, reference to an “antioxidant” is a reference to one or more antioxidants and equivalents thereof known to those skilled in the art, and so forth.

As used herein, the term “about” means plus or minus 10% of the numerical value of the number with which it is being used. Therefore, about 50% means in the range of 45%-55%.

The term “animal,” “patient,” or “subject” as used herein includes, but is not limited to, humans and non-human vertebrates such as wild, domestic and farm animals. Preferably, the term refers to humans.

As used herein, “antibody fragments” that may be used include Fab, Fab′, F(ab′)2, scFv, dsFv, ds-scFv, dimers, minibodies, diabodies, bispecific antibody fragments, multimers, and any combination thereof, and fragments from recombinant sources and/or produced in transgenic animals. The antibody or fragment may be from any species including mice, rats, rabbits, hamsters and humans. Chimeric antibody derivatives, i.e., antibody molecules that combine a non-human animal variable region and a human constant region are also contemplated within the scope of the invention. Chimeric antibody molecules can include, for example, humanized antibodies which comprise the antigen binding domain from an antibody of a mouse, rat, or other species, with human constant regions. Conventional methods may be used to make chimeric antibodies. It is expected that chimeric antibodies would be less immunogenic in a human subject than the corresponding non-chimeric antibody. The humanized antibodies can be further stabilized for example as described in WO 00/61635 and is incorporated by reference in its entirety.

As used herein, the phrase “anticancer agents” refers to compounds or treatments that are effective in treating or preventing cancer including, without limitation, chemical agents, other immunotherapeutics, cancer vaccines, anti-angiogenic compounds, certain cytokines, certain hormones, gene therapy, radiotherapy, surgery, and dietary therapy.

As used herein, the phrase “effective amount” means an amount effective, at dosages and for periods of time necessary to achieve the desired result. Effective amounts of an immunoconjugate may vary according to factors such as the disease state, age, sex, weight of the animal. Dosage regimen may be adjusted to provide the optimum therapeutic response. For example, several divided doses may be administered daily or the dose may be proportionally reduced as indicated by the exigencies of the therapeutic situation.

As used herein, the phrase “humanized antibody or antibody fragment” means that the antibody or fragment comprises human framework regions.

As used herein, the phrase “immunoconjugate” refers to an antibody conjugated to an effector molecule. In some embodiments, the antibody may be full length antibody or antibody fragments, such as Fab, Fab′, F(ab)₂, scFv, dsFv, ds-scFv, dimers, minibodies, diabodies, bispecific antibody fragments, multimers, and any combination thereof, and fragments from recombinant sources and/or produced in transgenic animals. In some embodiments, the antibody may be a synthetic protein, a binding protein or a polypeptide. In some embodiments, the effector molecule may be a toxin, a radionucleotide, a radiopharmaceutical, a labeling agent, a drug, a cytotoxic agent, a peptide, a protein and the like. These efector molecules may be capable of killing, lysing or labeling or inducing other effects when the antibody binds to an antigen.

As used herein, the phrase “is administered directly to the cancer site or direct administration” refers to direct or substantially direct introduction including, without limitation, single or multiple injections of the immunoconjugate directly into the tumor or peritumorally, continuous or discontinuous perfusion into the tumor or peritumorally, introduction of a reservoir into the tumor or peritumorally, introduction of a slow-release apparatus into the tumor or peritumorally, introduction of a slow-release formulation into the tumor or peritumorally, direct application onto the tumor, direct injection into an artery that substantially directly feeds the area of the tumor, direct injection into a lymphatic vessel that substantially drains into the area of the tumor, direct or substantially direct introduction in a substantially enclosed cavity (e.g., pleural cavity) or lumen (e.g., intravesicular). “Peritumoral” is a term that describes a region, within about 10 cm, preferably within 5 cm, more preferably within 1 cm, of what is regarded as the tumor boundary, such as, but not limited to, a palpable tumor border. “Direct administration” in the context of prevention of occurrence or prevention of recurrence is defined as administration directly into a site at risk for development or recurrence of a cancer.

As used herein, the term “MOC-31 antibody” means the murine anti-Ep-CAM or anti-EGP-2 antibody and is available from commercial sources such as BioGenex, cat No. MU316-UC, Zymed Laboratories Inc., cat. No. 18-0270 or United States Biological, cat No. M4165.

As used herein, the term “4D5MOC-A” means the humanized scFv MOC31 antibody grafted onto the artificial human consensus framework of scFv 4D5 as described in WO 00/61635 which is incorporated herein by reference in its entirety.

As used herein, the term “4D5MOC-B” means a stable variant of 4D5MOC-A prepared as described in WO 00/61635 which is incorporated herein by reference in its entirety.

As used herein, the term “VB4-845” means an immunoconjugate that comprises a) the scFv humanized antibody 4D5MOC-B that is fused to b) a truncated form of Pseudomonas exotoxin A (amino acids 252-608). Details of VB4-845 have been disclosed in US20100249039 which is incorporated herein by reference.

As used herein, the phrase “pharmaceutically acceptable” refers to general clinical use and/or approval by a regulatory agency of the Federal or state government, listing in the United States Pharmacopoeia, or general acceptance by those skilled in the relevant art.

As used herein, “physiologic conditions” for antibody binding reflect but do not necessarily exactly duplicate the conditions in which an Ep-CAM-binding polypeptide would encounter an Ep-CAM molecule in vivo. Binding under physiologic conditions should be reasonably predictive that binding in vivo will occur.

As used herein, the phrase “preventing cancer” refers to prevention of cancer occurrence. In certain instances, the preventative treatment reduces the recurrence of the cancer. In other instances, preventative treatment decreases the risk of a patient from developing a cancer, or inhibits progression of a pre-cancerous state (e.g., a colon polyp) to actual malignancy.

As used herein, the phrase “reduced dose” refers to a dose that is below the normally administered and/or recommended dose. The normally administered dose of an anticancer agent can be found in reference materials well known in the art such as, for example, the latest edition of the Physician's Desk Reference.

As used herein, the phrase “treating cancer” refers to inhibition of cancer cell replication, apoptosis, inhibition of tumor growth, reduction of cancer cell number or tumor growth, decrease in the malignant grade of a cancer (e.g., increased differentiation), or improved cancer-related symptoms.

As used herein, the term “therapeutic” means an agent utilized to discourage, combat, ameliorate, prevent or improve an unwanted condition, disease or symptom of a patient.

As used herein, the term “variant” refers to any pharmaceutically acceptable derivative, analogue, or fragment of an immunoconjugate, an antibody or antibody fragment, a toxin (e.g., Pseudomonas toxin), or an effector molecule described herein. A variant also encompasses one or more components of a multimer, multimers comprising an individual component, multimers comprising multiples of an individual component (e.g., multimers of a reference molecule), a chemical breakdown product, and a biological breakdown product. In particular, non-limiting embodiments, an immunoconjugate may be a “variant” relative to a reference immunoconjugate by virtue of alteration(s) in the Ep-CAM-binding portion and/or the toxin portion of the reference immunoconjugate. For example, a variant immunoconjugate may contain multimers of the antibody portion and/or the toxin portion. A variant of the toxin portion of the molecule retains toxicity of at least 10%, at least 30%, at least 50%, at least 80%, at least 90%, in a standard assay used to measure toxicity of a preparation of the reference toxin. In some embodiments, a variant may also refer to polypeptides having at least 50%, at least 60%, at least 70%, at least 80%, at least 90%, or 95% sequence identity to the immunoconjugate of the present invention. In some embodiments, a variant may also refer to a polypeptides or proteins having at least 30%, at least 60%, at least 70%, at least 80%, at least 90%, or 95% binding affinity of the immunoconjugate of the present invention, when measured by a competitive binding assay.

A variant immunoconjugate having a variation of the Ep-CAM-binding portion of the reference immunoconjugate competes with the binding of an anti-Ep-CAM reference antibody, under physiologic conditions, by at least 10 percent and preferably at least 30 percent (and see infra). Competition by 10 percent means that, in an assay where a saturating concentration of anti-Ep-CAM reference antibody is bound to Ep-CAM, 10 percent of these bound reference antibodies is displaced when an equilibrium is reached with an equivalent concentration of the variant anti-Ep-CAM immunoconjugate being tested. As a non-limiting example, competition between antibodies, or between an antibody and an immunoconjugate, is measured by: binding labeled anti-Ep-CAM reference antibody to Ep-CAM on the surface of cells, or to an Ep-CAM-coated solid substrate, such that virtually all Ep-CAM sites are bound by the antibody; contacting these antibody-antigen complexes with unlabeled test anti-Ep-CAM antibody or unlabeled test immunoconjugate; and measuring the amount of labeled antibody displaced from Ep-CAM binding sites, wherein the amount of freed, labeled antibody indicates the amount of competition that has occurred.

Immunotherapy has emerged as a powerful tool to combat cancer. Murine and humanized/chimeric antibodies, and their respective antibody fragments, directed against tumor-associated antigens (“TAAs”) have been used for diagnosis and therapy of certain human cancers. Unconjugated, toxin-conjugated, and radiolabeled forms of these antibodies have been used in such therapies.

One tumor associated antigen of interest for immunotherapy is Epithelial Cell Adhesion Molecule (“Ep-CAM”) which also known as 17-1 A, KSA, EGP-2 and GA733-2. Ep-CAM is a transmembrane protein that is highly expressed in many solid tumors, including carcinomas of the lung, breast, ovary, colorectum, and squamous cell carcinoma of the head and neck, but weakly expressed in most normal epithelial tissues. Its expression correlates with the rate of cellular proliferation. Ep-CAM-specific antibodies have been used to image and detect primary tumors in patients with small cell lung cancer and non-small cell lung cancer.

Hepatocellular carcinoma (“HCC”) is the fifth most common cancer and one of the leading causes of cancer death worldwide. HCC has a poor prognosis and the 5-year survival rate of HCC has remained below 12% in the United States. The malignant potential of HCC tumors has been reported in respect to several histopathological findings including vascular invasion and gross morphology. Although the primary curative treatment for HCC is surgical resection including liver transplantation, various therapeutic options have been employed including radiofrequency ablation, transarterial chemoembolization, and chemotherapy (5-FU). Effective palliative treatment is hindered by the fact that HCC is frequently resistant to conventional cytotoxic agents. The median overall survival among patients with advanced HCC is still less than 1 year and the prognosis remains poor.

Many cancer cells become resistant to current therapies of chemotherapy and radiation, and a small group of cells persist even after extensive treatment. One hypothesis to explain this resistance is the presence of cancer stem cells. Not wishing to be bound by theory, a distinct subset of cells within each tumor are capable of indefinite self-renewal and can develop into adult tumor cell(s), which are relatively limited in replication capacity. It has been hypothesized that these cancer stem cells (CSC) might be more resistant to chemotherapeutic agents, radiation or other toxic conditions, and thus, persist after clinical therapies and later grow into secondary tumors, metastases or be responsible for relapse. It has been suggested that CSCs can arise either from the tissue stem cells or from a more differentiated tissue progenitor cell(s).

Some researchers have proposed that cancer stem cells can be identified based on marker expression. For example, CD133 has been proposed to be a marker found in cancer stem cells in brain tumors and in human prostatic epithelial stem cells. CD44 expression accompanied by no or low CD24 expression is expressed by some breast cancer stem cells. Colon cancer stem cells express CD133, CD44, and CD166. Hepatic stem cell markers include Ep-CAM, CD133, CD44, and CD90.

In response to this medical problem, there is considerable need for the development of new, tumor-specific therapies. One novel approach is targeted therapy using an immunoconjugate, such as an antibody conjugated with a toxin. The antibody binds specifically to tumor cells to deliver the toxin for efficient tumor cell-killing.

It is disclosed herein that an immunoconjugate comprising a humanized antibody fragment that binds to the extracellular domain of human Ep-CAM linked to Pseudomonas exotoxin A is effective in treating hepatocellular carcinoma. In particular, the inventors have shown that an immunoconjugate comprising a single-chain Fv recombinant stabilized and humanized antibody fragment to Ep-CAM that has been fused to a truncated form of Pseudomonas exotoxin A (ETA) which lacks the cell binding domain is cytotoxic against liver cancer cells. This immunoconjugate binds to Ep-CAM expressed on the liver cancer cells. Once bound, the immunoconjugate is internalized and the Pseudomonas exotoxin A kills cells or blocks the protein synthesis, thereby leading to cell death. Importantly, since most normal mucosal cells and fibroblasts do not widely express Ep-CAM, and therefore cannot internalize the immunoconjugate, they are protected from the potential side-effects of the exotoxin.

The present disclosure is related to compositions and methods for treating hepatocellular carcinoma. In one embodiment, a method of treating a subject with hepatocellular carcinoma comprises administering to said subject a therapeutically effective amount of an immunoconjugate comprising an antibody conjugated to an effector molecule, and wherein the antibody recognizes epithelial cell adhesion molecule (Ep-CAM). The antibody comprises light chain complementarity determining regions (CDRs) comprising the amino acid sequences defined by SEQ ID NOS: 4, 5 and 6, and heavy chain complementarity determining regions comprising the amino acid sequences defined by SEQ ID NOS: 7, 8, and 9. The effector molecule may be radioisotopes, antineoplastic agents, immunomodulators, biological response modifiers, lectins, toxins, and any combination thereof. In some embodiments, the effector molecule may be a toxin, such as abrin, modeccin, viscumin, gelonin, bouganin, saporin, ricin, ricin A chain, bryodin, luffin, momordin, restrictocin, Pseudomonas exotoxin A, pertussis toxin, tetanus toxin, botulinum toxin, Shigella toxin, cholera toxin, diphtheria toxin and any combination thereof. In some embodiments, the immunoconjugate is administered directly to the cancer site.

In an additional embodiment, the immunoconjugate is VB4-845 as shown in SEQ ID NO: 2, or a variant thereof. In some embodiments, the immunoconjugate may be co-administered, concurrently administered, or sequentially administered with one or more other anticancer agents. In some embodiments, the immunoconjugate VB4-845 may lack the pelB leader sequence, and comprises an amino acid sequence from amino acid 23 to amino acid 669 of SEQ ID NO: 2.

In another embodiment, a method of detecting or monitoring hepatocellular carcinoma in a subject includes the steps of: contacting a test sample taken from said subject with an antibody to form an antibody-antigen complex, wherein the antibody comprises light chain complementarity determining regions comprising the amino acid sequences defined by SEQ ID NOS: 4, 5 and 6, and heavy chain complementarity determining regions comprising the amino acid sequences defined by SEQ ID NOS: 7, 8, and 9; measuring the amount of antibody-antigen complex in the test sample; and normalizing the results against a control.

In a further embodiment, a kit for diagnosing hepatocellular carcinoma comprises an antigen comprising light chain complementarity determining regions comprising the amino acid sequences defined by SEQ ID NOS: 4, 5 and 6, and heavy chain complementarity determining regions comprising the amino acid sequences defined by SEQ ID NOS: 7, 8, and 9; and instructions for the use thereof.

In an additional embodiment, a method for treating hepatocellular carcinoma involves: testing a tumor sample from a patient for the expression of Epithelial Cell Adhesion Molecule (Ep-CAM); and if the protein is expressed at greater levels in the tumor sample as compared to a control, administering to the patient an effective amount of VB4-845 having the sequence shown in SEQ ID NO: 2. In some embodiments, the immunoconjugate VB4-845 may lack the pelB leader sequence, and comprises an amino acid sequence from amino acid 23 to amino acid 669 of SEQ ID NO: 2. In some embodiments, the immunoconjugate is administered directly to the cancer site.

In a further embodiment, a kit for treating hepatocellular carcinoma comprises an effective amount of an immunoconjugate, wherein the immunoconjugate is VB4-845 having the sequence shown in SEQ ID NO: 2 and directions for the use thereof to treat the cancer. In some embodiments, the immunoconjugate VB4-845 may lack the pelB leader sequence, and comprises an amino acid sequence from amino acid 23 to amino acid 669 of SEQ ID NO: 2.

The methods and systems disclosed herein are contemplated to treat primary tumors in liver or hepatocellular carcinoma, or to decrease metastatic potential of the hepatocellular carcinoma. Such methods do not include treating liver metastases, wherein cancerous tumors of different origin metastasize (spread) from another part of the body to the liver.

In another embodiment, a method of killing cancer stem cells in vitro or in vivo comprises contacting the cancer stem cells to an effective amount of an immunoconjugate comprising an antibody conjugated to an effector molecule, and wherein the antibody recognizes epithelial cell adhesion molecule (Ep-CAM). Many malignant tumors with poor prognosis show preferential overexpression of genes that are normally enriched in embryonic stem cells. Some of these hepatic stem/progenitor markers include Ep-CAM, CD133, CD44, and CD90. So, cancer cells expressing stem/progenitor markers might be recognized as the critical targets for the treatment of hepatocellular carcinoma. In addition, the immunoconjugates disclosed herein may also be used to kill a variety of cancer stem cells, such as lung cancer stem cells, breast cancer stem cells, prostate cancer stem cells, liver cancer stem cells, brain cancer tem cells, bladder cancer stem cells, colon cancer stem cells, gastric cancer stem cells, head and neck cancer stem cells, pancreatic cancer stem cells, and ovarian cancer stem cells.

In an additional embodiment, a method of killing liver cancer cells in vitro or in vivo involves contacting the liver cancer cells to an effective amount of an immunoconjugate comprising an antibody conjugated to an effector molecule, and wherein the antibody recognizes epithelial cell adhesion molecule (Ep-CAM).

In some embodiments, a method of killing liver cancer cells in vitro or in vivo include contacting liver cancer cells with an effective amount of an immunoconjugate along with an anticancer agent. The immunoconjugate may comprise an antibody conjugated to an effector molecule, and wherein the antibody recognizes epithelial cell adhesion molecule (Ep-CAM). The anticancer agent may be any anticancer agent described herein. In some embodiments, the immunoconjugate is VB4-845 and the anticancer agent is 5-fluorouracil.

Accordingly, in one embodiment, the present invention provides a method for treating or preventing hepatocellular carcinoma comprising administering to an animal in need of such treatment an effective amount of an immunoconjugate comprising: (a) an antibody that binds to a protein on the cancer cell attached to; (b) a toxin that is cytotoxic to the cancer cells. The present invention also provides an use of an effective amount of an immunoconjugate comprising: (a) an antibody that binds to a protein on the cancer cell attached to; (b) a toxin that is cytotoxic to the cancer cells to treat or prevent hepatocellular carcinoma. The present invention further provides an use of an effective amount of an immunoconjugate comprising: (a) an antibody that binds to a protein on the cancer cell attached to; (b) a toxin that is cytotoxic to the cancer cells in the manufacture of a medicament to treat or prevent hepatocellular carcinoma.

In another embodiment, the present invention provides a method for killing cancer stem cells comprising administering to an animal in need of such treatment an effective amount of an immunoconjugate comprising: (a) an antibody that binds to a protein on the cancer cell attached to; (b) a toxin that is cytotoxic to the cancer cells. The present invention also provides an use of an effective amount of an immunoconjugate comprising: (a) an antibody that binds to a protein on the cancer cell attached to; (b) a toxin that is cytotoxic to the cancer stem cells. The present invention further provides an use of an effective amount of an immunoconjugate comprising: (a) an antibody that binds to a protein on the cancer cell attached to; (b) a toxin that is cytotoxic to the cancer cells in the manufacture of a medicament to kill cancer stem cells.

The antibody that binds to a protein on the cancer cell can be any molecule that can selectively target the immunoconjugate to the cancer cells. In one embodiment, the antibody binds to a tumor associated antigen. Examples of proteins that are expressed on liver cancer cells include IL-4 receptor, the EGF-receptor, the HER2/neu surface protein, EGF-receptor, gp54, Ep-CAM, CD133, CD13, CD44, and CD90. In a specific embodiment, the antibody binds to Ep-CAM.

Specific antibodies, or antibody fragments that recognize antigens on liver cancer cells or cancer stem cells may also be generated by screening expression libraries encoding immunoglobulin genes, or portions thereof, expressed in bacteria with peptides produced from the nucleic acid molecules encoding the proteins. For example, complete Fab fragments, V_{H} regions and Fᵥ regions can be expressed in bacteria using phage expression libraries. Alternatively, a SCID-hu mouse can be used to produce antibodies or fragments thereof.

The antibody portion of an immunoconjugate may be immunoglobulin derived, i.e., can be traced to a starting molecule that is an immunoglobulin (or antibody). For example, the antibody may be produced by modification of an immunoglobulin scaffold using standard techniques known in the art. In another, non-limiting example, immunoglobulin domains (e.g., variable heavy and/or light chains) may be linked to a non-immunoglobulin scaffold. Further, the antibody may be developed by, without limitation, chemical reaction or genetic design. Accordingly, in a non-limiting example, an immunoconjugate may comprise: an immunoglobulin-derived polypeptide (e.g., an antibody selected from an antibody library), or variant thereof, that specifically binds to liver cancer cells; and a toxin or variant thereof. Such immunoglobulin polypeptides can be re-designed to affect their binding characteristics to a target a tumor associated molecule, or to improve their physical characteristics, for example.

The antibody portion of the immunoconjugate need not be immunoglobulin based. Accordingly, an immunoconjugate may comprise: a non-immunoglobulin polypeptide (e.g., Affibody®), or variant thereof, that specifically binds to liver cancer cells; and a toxin or variant thereof. Such non-immunoglobulin polypeptide can be designed to bind to a target tumor associated molecule. Moreover, non-immunoglobulin polypeptide can be engineered to a desired affinity or avidity, and can be designed to tolerate a variety of physical conditions, including extreme pH ranges and relatively high temperature.

Indeed, for use in a pharmaceutical composition, the design of a non-immunoglobulin polypeptide with a relatively long half-life at physiological conditions (e.g., 37° C. in the presence of peptidases) can be advantageous. Furthermore, such molecules, or variants thereof, may demonstrate good solubility, small size, proper folding and can be expressed in readily available, low-cost bacterial systems, and thus manufactured in commercially reasonable quantities. The ability to design a non-immunoglobulin polypeptide is within the skill of the ordinary artisan.

Examples of epitope-binding polypeptides include, without limitation, ligands comprising a fibronectin type III domain, binding molecules based on assembly of repeat protein domains comprising Pleckstrin-Homology (PH) domains, ankyrin repeats, and the like.

In some embodiments, the immunioconjugate may be a humanized, stabilized, single-chain, anti-Ep-CAM antibody, 4D5MOC-B, which is derived from murine monoclonal antibody MOC31, and is the subject of this invention.

In some embodiments, the antibody preferably recognizes Ep-CAM. In one embodiment, the immunoconjugate comprises (a) an antibody or antibody fragment that binds to Ep-CAM on the cancer cell attached to; (b) a toxin that is cytotoxic to the cancer cells. In a specific embodiment, the immunoconjugate comprises (a) a humanized antibody or antibody fragment that binds to the extracellular domain of human Ep-CAM and comprises complementarity determining region (CDR) sequences derived from a MOC-31 antibody attached to; (b) a toxin that is cytotoxic to the cancer cells. CDR sequences from the 4D5MOC-B antibody are shown in SEQ ID NOS:4-9.

In one embodiment, the variant amino acid sequences of the light chain CDR1, CDR2 and CDR3, and the heavy chain CDR1, CDR2 and CDR3 have at least 50%, preferably at least 60%, more preferably at least 70%, most preferably at least 80%, even more preferably at least 90%, and even most preferably 95% sequence identity to SEQ ID NOS: 4-9, respectively.

In another embodiment, the variant amino acid sequences of the light chain variable region and the heavy chain variable region of Ep-CAM antibody have at least 50%, preferably at least 60%, more preferably at least 70%, most preferably at least 80%, even more preferably at least 90% and even most preferably 95% sequence identity to SEQ ID NO:1.

Suitable Ep-CAM-targeted immunoconjugates include, without limitation, VB4-845 and variants thereof, other immunoconjugates that comprise the MOC31 variable region or variants thereof, as well as immunoconjugates that comprise other single or double chain immunoglobulins that selectively bind Ep-CAM, or variants thereof.

In a specific, non-limiting embodiment, the immunoconjugate comprises VB4-845 as shown in SEQ ID NO: 2. In other non-limiting embodiments, the immunoconjugate comprises a variant of VB4-845. A VB4-845 variant binds to the same Ep-CAM epitope or to a substantially similar Ep-CAM epitope that is bound by VB4-845, and the variant may competitively inhibit VB4-845 binding to Ep-CAM, under physiologic conditions, by at least 10%, 15%, 20%, 25%, 30%, 35%, 40%, 45%, 50%, 55%, 60%, 65%, 70%, 75%, 80%, 85%, 90%, or 95%. A VB4-845 variant may comprise the same Pseudomonas exotoxin A fragment as VB4-845, or may comprise a different portion of the same exotoxin or a different toxin. In some embodiments, the immunoconjugate VB4-845 may lack the pelB leader sequence, and comprises an amino acid sequence from amino acid 23 to amino acid 669 of SEQ ID NO: 2.

In one embodiment, the variant amino acid sequences of VB4-845 have at least 50%, preferably at least 60%, more preferably at least 70%, most preferably at least 80%, even more preferably at least 90%, and even most preferably at least 95% sequence identity to SEQ ID NO: 2.

Likewise, a variety of toxins may be used to design an Ep-CAM-targeted immunoconjugate according to the invention. In preferred embodiments, the toxins may be plant toxins or bacterial toxins. Non-limiting examples include abrin, modeccin, viscumin, gelonin, bouganin, saporin, ricin, ricin A chain, bryodin, luffin, momordin, restrictocin, Pseudomonas exotoxin A, pertussis toxin, tetanus toxin, botulinum toxin, Shigella toxin, cholera toxin, diphtheria toxin and combinations thereof. When the toxin is a ribosome-inactivating protein, the immunoconjugate may be internalized upon binding to the cancer cell in order for the toxin to be cytotoxic to the cells.

In a particular preferred embodiment, the toxin portion comprises at least a toxic portion of Pseudomonas exotoxin A (“ETA”), or a variant thereof. In a specific embodiment, the cytotoxic portion comprises an ETA variant that, when administered alone, is substantially unable to bind to cells. In a further, specific embodiment, the cytotoxic portion comprises ETA₂₅₂₋₆₀₈. The cytotoxic portion may comprises one or more Pseudomonas exotoxins known in the art.

In other non-limiting embodiments, the toxin comprises an agent that acts to disrupt DNA. Thus, toxins may comprise, without limitation, enediynes (e.g., calicheamicin and esperamicin) and non-enediyne small molecule agents (e.g., bleomycin, methidiumpropyl-EDTA-Fe(II)). Other toxins useful in accordance with the invention include, without limitation, daunorubicin, doxorubicin, distamycin A, cisplatin, mitomycin C, ecteinascidins, duocarmycin/CC-1065, and bleomycin/pepleomycin.

In other non-limiting embodiments, the toxin comprises an agent that acts to disrupt tubulin. Such toxins may comprise, without limitation, rhizoxin/maytansine, paclitaxel, vincristine and vinblastine, colchicine, auristatin dolastatin 10 MMAE, and peloruside A.

In other non-limiting embodiments, the toxin portion of an immunoconjugate of the invention may comprise an alkylating agent including, without limitation, Asaley NSC 167780, AZQ NSC 182986, BCNU NSC 409962, Busulfan NSC 750, carboxyphthalatoplatinum NSC 271674, CBDCA NSC 241240, CCNU NSC 79037, CHIP NSC 256927, chlorambucil NSC 3088, chlorozotocin NSC 178248, cisplatinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762, PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In other non-limiting embodiments, the toxin portion of an immunoconjugate of the invention may comprise an antimitotic agent including, without limitation, allocolchicine NSC 406042, Halichondrin B NSC 609395, colchicine NSC 757, colchicine derivative NSC 33410, dolastatin 10 NSC 376128, maytansine NSC 153858, rhizoxin NSC 332598, taxol NSC 125973, taxol derivative NSC 608832, thiocolchicine NSC 361792, trityl cysteine NSC 83265, vinblastine sulfate NSC 49842, and vincristine sulfate NSC 67574.

In other non-limiting embodiments, the toxin portion of an immunoconjugate of the invention may comprise an topoisomerase I inhibitor including, without limitation, camptothecin NSC 94600, camptothecin, Na salt NSC 100880, aminocamptothecin NSC 603071, camptothecin derivative NSC 95382, camptothecin derivative NSC 107124, camptothecin derivative NSC 643833, camptothecin derivative NSC 629971, camptothecin derivative NSC 295500, camptothecin derivative NSC 249910, camptothecin derivative NSC 606985, camptothecin derivative NSC 374028, camptothecin derivative NSC 176323, camptothecin derivative NSC 295501, camptothecin derivative NSC 606172, camptothecin derivative NSC 606173, camptothecin derivative NSC 618939, camptothecin derivative NSC 610457, camptothecin derivative NSC 606499, camptothecin derivative NSC 610456, camptothecin derivative NSC 364830, camptothecin derivative NSC 606497, and morpholinodoxorubicin NSC 354646.

In other non-limiting embodiments, the toxin portion of an immunoconjugate of the invention may comprise an topoisomerase II inhibitor including, without limitation, doxorubicin NSC 123127, amonafide NSC 308847, m-AMSA NSC 249992, anthrapyrazole derivative NSC 355644, pyrazoloacridine NSC 366140, bisantrene HCL NSC 337766, daunorubicin NSC 82151, deoxydoxorubicin NSC 267469, mitoxantrone NSC 301739, menogaril NSC 269148, N,N-dibenzyl daunomycin NSC 268242, oxanthrazole NSC 349174, rubidazone NSC 164011, VM-26 NSC 122819, and VP-16 NSC 141540.

In other non-limiting embodiments, the toxin portion of an immunoconjugate of the invention may comprise an RNA or DNA antimetabolite including, without limitation, L-al-anosine NSC 153353, 5-azacytidine NSC 102816, 5-fluorouracilNSC 19893, acivicin NSC 163501, aminopterin derivative NSC 132483, aminopterin derivative NSC 184692, aminopterin derivative NSC 134033, an antifol NSC 633713, anantifol NSC 623017, Baker's soluble antifol NSC 139105, dichlorallyl lawsone NSC 126771, brequinar NSC 368390, ftorafur (pro-drug) NSC 148958, 5,6-dihydro-5-azacytidine NSC 264880, methotrexate NSC 740, methotrexate derivative NSC 174121, N-(phosphonoacetyl)-L-aspartate (PALA) NSC 224131, pyrazofurin NSC 143095, trimetrexate NSC 352122, 3-HP NSC 95678, 2′-deoxy-5-fluorouridine NSC 27640, 5-HPNSC 107392, alpha-TGDR NSC 71851, aphidicolin glycinate NSC 303812, ara-C NSC 63878, 5-aza-2′-deoxycytidine NSC 127716, beta-TGDRNSC 71261, cyclocytidine NSC 145668, guanazole NSC 1895, hydroxyurea NSC 32065, inosine glycodialdehyde NSC 118994, macbecin II NSC 330500, pyrazoloimidazole NSC 51143, thioguanine NSC 752, and thiopurine NSC 755.

The antibody may be conjugated to the target by any means by which the antibody can be associated with, or linked to, the toxin. For example, the antibody or the antibody fragment may be attached to the toxin by chemical or recombinant means. Chemical means for preparing fusions or conjugates are known in the art and can be used to prepare the immunoconjugate. The method used to conjugate the antibody and toxin must be capable of joining the antibody with the toxin without interfering with the ability of the antibody to bind to the target molecule on the cancer cell.

In one embodiment, the antibody and toxin are both proteins and can be conjugated using techniques well known in the art. There are several hundred crosslinkers disclosed in the art that can conjugate two proteins. The crosslinker is generally chosen based on the reactive functional groups available or inserted on the antibody or toxin. In addition, if there are no reactive groups, a photoactivatible crosslinker can be used. In certain instances, it may be desirable to include a spacer between the antibody and the toxin. Crosslinking agents known to the art include the homobifunctional agents: glutaralde-hyde, dimethyladipimidate and bis(diazobenzidine) and the heterobifunctional agents: m-maleimidobenzoyl-N-hydroxysuccinimide and sulfo-m maleimidobenzoyl-N-hydroxysuccinimide.

A antibody-toxin protein fusion may also be prepared using recombinant DNA techniques. In such a case a DNA sequence encoding the antibody is fused to a DNA sequence encoding the toxin, resulting in a chimeric DNA molecule. The chimeric DNA sequence is transfected into a host cell that expresses the antibody-toxin fusion protein. The fusion protein can be recovered from the cell culture and purified using techniques known in the art.

In some embodiments, the immunoconjugate of the present invention can be used to treat liver cancer or hepatocellular carcinoma.

In addition, the present invention also provides methods to kill cancer stem cells, including hepatocytes expressing stem/progenitor markers. Tumors or tumor cells may be evaluated to determine their susceptibility to the treatment methods of the invention by, for example, obtaining a sample of tumor tissue or cells and determining the ability of the sample to bind to the antibody portion of the immunoconjugate. In one embodiment, the protein on the cancer cells is Ep-CAM. Cell-surface expression of Ep-CAM may be induced, or elevated, by an agent that increases steady-state levels of cell-surface Ep-CAM in pre-cancerous or cancerous tissue.

Accordingly, the present invention includes diagnostic methods and kits that can be used prior to the therapeutic method of the invention in order to determine whether or not the liver cancer cells expresses levels of the protein that are bound by the antibody in the immunoconjugate. Therefore, in a further embodiment, the present invention includes a method for treating or preventing hepatocellular carcinoma comprising: testing a tumor sample from a patient for the expression of Epithelial Cell Adhesion Molecule (Ep-CAM); and if the protein is expressed at greater levels in the tumor sample as compared to a control, administering to the patient an effective amount of VB4-845 having the sequence shown in SEQ ID NO: 2.

The present invention further includes a kit for diagnosing hepatocellular carcinoma comprising an antibody that binds to a protein on the cancer cell and instructions for the use thereof to diagnose the cancer.

In preferred non-limiting embodiments, the cancer is amenable to treatment by direct administration of the immunoconjugate. For example, a target tumor mass may be close to the surface of the skin. In another example, a diseased tissue may be encapsulated by a cyst, or is found in a substantially enclosed cavity including, without limitation, a lumen. In other embodiments, the cancer is amenable to treatment by intravenous administration of the immunoconjugate.

The invention also provides methods for reducing the risk of post-surgical complications comprising administering an effective amount of an immunoconjugate before, during, or after surgery, and in specific non-limiting embodiments, surgery to treat cancer.

The invention also provides methods for preventing occurrence, preventing or delaying recurrence, or reducing the rate of recurrence of hepatocellular carcinoma comprising directly administering to a patient in need thereof an effective amount of an immunoconjugate.

The invention also provides methods for sensitizing a tumor or cancer to one or more other anticancer agents comprising administering an immunoconjugate of the invention. In a non-limiting embodiment, the other anticancer agent comprises another Ep-CAM-targeted immunoconjugate. In another non-limiting embodiment, the other anticancer agent comprises radiation. The other anticancer agents may be administered prior to, overlapping with, concurrently, and/or after administration of the immunoconjugate. When administered concurrently, the immunoconjugate and other anticancer agent may be administered in a single formulation or in separate formulations, and if separately, then optionally, by different modes of administration. Accordingly, the combination of one or more immunoconjugates and one or more other anticancer agents may synergistically act to combat the tumor or cancer.

In some embodiments, the anticancer agents may be tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, vincristine, taxol, taxotere, etoposide, teniposide, amsacrine, Irinotecan, topotecan, an epothilone, gefitinib, erlotinib, sorafenib, angiogenesis inhibitors, EGF inhibitors, VEGF inhibitors, CDK inhibitors, cytokines, Her1 and Her2 inhibitors, and monoclonal antibodies.

In another embodiment, an immunoconjugate is administered in combination with a regimen of radiation therapy. The therapy may also comprise surgery and/or chemotherapy. For example, the immunoconjugate may be administered in combination with radiation therapy and cisplatin (Platinol), fluo-rouracil (5-FU, Adrucil), carboplatin (Paraplatin), and/or paclitaxel (Taxol). Treatment with the immunoconjugate may allow use of lower doses of radiation and/or less frequent radiation treatments, which may for example, reduce the incidence of severe sore throat that impedes swallowing function potentially resulting in undesired weight loss or dehydration.

Where an immunoconjugate of the invention is administered in addition to one or more other anticancer agents, these other anticancer agents may include, without limitation, 2,2′,2″trichlorotriethylamine, 6-azauridine, 6-diazo-5-oxo-L-norleucine, mercaptopurine, aceglarone, aclacinomycinsa actinomycin, altretamine, aminoglutethimide, amsacrine, anastrozole, ancitabine, angiogenin antisense oligonucleotide, anthramycin, azacitidine, azaserine, aziridine, batimastar, bcl-2 antisense oligonucleotide, benzodepa, bicalutamide, bisantrene, bleomycin, buserelin, busulfan, cactinomycin, calusterone, carboplatin, carboquone, carmofur, carmustine, carubicin, carzinophilin, chlorambucil, chloraphazine, chlormadinone acetate, chlorozotocin, chromomycins, cisplatin, cladribine, cyclophosphamide, cytarabine, dacarbazine, dactinomycin, daunorubicin, defosfamide, demecolcine, denopterin, diaziquone, docetaxel, doxifluridine, doxorubicin, droloxifene, dromo-stanolone, edatrexate, eflornithine, elliptinium acetate, emitefur, enocitabune, epirubicin, epitiostanol, estramustine, etoglucid, etoposide, fadrozole, fenretinide, floxuridine, fludarabine, fluorouracil, flutamide, folinic acid, formestane, fosfestrol, fotemustine, gallium nitrate, gemcitabine, goserelin, hexestrol, hydroxyurea, idarubicin, ifosfamide, improsulfan, interferonalpha, interferonbeta, interferon-gamma, interleukin-2, L-asparaginase, lentinan, letrozole, leuprolide, lomustine, lonidamine, mannomustine, mechlorethamine, mechlorethamine oxide hydrochloride, medroxyprogesterone, megestrol acetate, melengestrol, melphalan, menogaril, mepitiostane, methotrexate, meturedepa, miboplatin, miltefosine, mitobronitol, mitoguazone, mitolactol, mitomycins, mitotane, mitoxantrone, mopidamol, mycophenolic acid, nilutamide, nimustine, nitracine, nogalamycin, novembichin, olivomycins, oxaliplatin, paclitaxel, pentostain, peplomycin, perfosfamide, phenamet, phenesterine, pipobroman, piposulfan, pirarubicin, piritrexim, plicamycin, podophyllinic acid 2-ethyl-hydrazide, polyestradiol phosphate, porfimer sodium, porfiromycin, prednimustine, procabazine, propagermanium, PSK, pteropterin, puromycin, ranimustine, razoxane, roquinimex, sizofican, sobuzoxane, spirogerma-nium, streptonigrin, streptozocin, tamoxifen, tegafur, temozolomide, teniposide, tenuzonic acid, testolacone, thiamiprine, thioguanine, Tomudex, topotecan, toremifene, triaziquone, triethylenemelamine, triethylenephosphoramide, triethylenethiophosphoramide, trilostane, trimetrexate, triptorelin, trofosfamide, trontecan, tubercidin, ubenimex, uracil mustard, uredepa, urethan, vincristine, zinostatin, zorubicin, cytosine arabinoside, gemtuzumab, thioepa, cyclothosphamide, antimetabolites (e.g., methotrexate, 6-mercaptopurine, 6-thioguanine, cytarabine, 5-fluorouracil, fludarabine, gemcitabine, dacarbazine, temozoamide), hexamethylmelamine, LYSODREN, nucleoside analogues, plant alkaloids (e.g., Taxol, paclitaxel, camptothecin, topotecan, irinotecan (CAMPTOSAR,CPT-11), vinca alkyloids such as vinblastine, podophyllotoxin, epipodophyllotoxin, VP-16 (etoposide), cytochalasin B, gramicidin D, ethidium bromide, emetine, anthracyclines, liposomal doxorubicin, dihydroxyanthracindione, mithramycin, actinomycin D, aldesleukin, allutamine, biaomycin, capecitabine, carboplain, chlorabusin, cyclarabine, daclinomycin, floxuridhe, lauprolide acetate, levamisole, lomusline, mercaptopurino, mesna, mitolanc, pegaspergase, pentoslatin, picamycin, riuxlmab, campath-1, straplozocin, tretinoin, VEGF antisense oligonucleotide, vindesine, and vinorelbine. Compositions comprising one or more anticancer agents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone. Likewise, the immunoconjugate of the invention may be used in conjunction with radiation therapy or other known anticancer modalities.

Pharmaceutical compositions for combination therapy may also include, without limitation, antibiotics (e.g., dactinomycin, bleomycin, mithramycin, anthramycin), asparaginase, BCG protein, diphtheria toxin, procaine, tetracaine, lidocaine, propranolol, anti-mitotic agents, abrin, ricin A, Pseudomonas exotoxin, nerve growth factor, platelet derived growth factor, tissue plasminogen activator, antihistaminic agents, anti-nausea agents, etc.

Indeed, direct administration of an effective amount of an immunoconjugate to a patient in need of such treatment may result in reduced doses of another anticancer agent having clinically significant efficacy. Such efficacy of the reduced dose of the other anticancer agent may not be observed absent administration with an immunoconjugate. Accordingly, the present invention provides methods for treating a tumor or cancer comprising administering a reduced dose of one or more other anticancer agents.

Moreover, combination therapy comprising an immunoconjugate to a patient in need of such treatment may permit relatively short treatment times when compared to the duration or number of cycles of standard treatment regimens. Accordingly, the present invention provides methods for treating a tumor or cancer comprising administering one or more other anticancer agents for relatively short duration and/or in fewer treatment cycles.

Thus, in accordance with the present invention, combination therapies comprising an immunoconjugate and another anticancer agent may reduce toxicity (i.e., side effects) of the overall cancer treatment. For example, reduced toxicity, when compared to a monotherapy or another combination therapy, may be observed when delivering a reduced dose of immunoconjugate and/or other anticancer agent, and/or when reducing the duration of a cycle (i.e., the period of a single administration or the period of a series of such administrations), and/or when reducing the number of cycles.

In a preferred embodiment, the invention provides methods for treating and/or ameliorating the clinical condition of patients suffering from hepatocellular carcinoma. Accordingly, the invention provides methods for: (i) decreasing the liver tumor size, growth rate, invasiveness, malignancy grade, and/or risk of recurrence; (ii) prolonging the disease-free interval following treatment; and (iii) decreasing metastatic potential of the hepatocellular carcinoma by administering to the patient an effective amount of an immunoconj ugate.

Clinical outcomes of cancer treatments using an immunoconjugate of the invention are readily discernible by one of skill in the relevant art, such as a physician. For example, standard medical tests to measure clinical markers of cancer may be strong indicators of the treatment's efficacy. Such tests may include, without limitation, physical examination, performance scales, disease markers, 12-lead ECG, tumor measurements, tissue biopsy, cytoscopy, cytology, longest diameter of tumor calculations, radiography, digital imaging of the tumor, vital signs, weight, recordation of adverse events, assessment of infectious episodes, assessment of concomitant medications, pain assessment, blood or serum chemistry, detecting serum markers, urinalysis, CT scan, and pharmacokinetic analysis. Furthermore, synergistic effects of a combination therapy comprising the immunoconjugate and another anticancer agent may be determined by comparative studies with patients undergoing monotherapy.

The effective dose of immunoconjugate to be administered during a cycle varies according to the mode of administration. Direct administration (e.g., intratumoral injection) requires much smaller total body doses of immunoconjugate as compared to systemic, intravenous administration of the immunoconjugate. It will be evident to the skilled artisan that local administration can result in lower body doses, and in those circumstances, and resulting low circulating plasma level of immunoconjugate would be expected and desired.

In one embodiment, the effective dose by direct administration of immunoconjugate may range from about 10 to 3000, 20 to 900, 30 to 800, 40 to 700, 50 to 600, 60 to 500, 70 to 400, 80 to 300, 90 to 200, or 100 to 150 micrograms/tumor/day. In other embodiments, the dose may range from approximately 10 to 20, 21 to 40, 41 to 80, 81 to 100, 101 to 130, 131 to 150, 151 to 200, 201 to 280, 281 to 350, 351 to 500, 501 to 1000, 1001 to 2000, or 2001 to 3000 micrograms/tumor/day. In specific embodiments, the dose may be at least approximately 20, 40, 80, 130, 200, 280, 400, 500, 750, 1000, 2000, or 3000 micrograms/tumor/day.

In another embodiment, the effective dose of immunoconjugate may range from about 100 to 5000, 200 to 4000, 300 to 3000, 400 to 2000, 500 to 1000, 600 to 900, or 700 to 1500 micrograms/tumor/month. In other embodiments, the dose may range from approximately 100 to 199, 200 to 399, 400 to 649, 650 to 999, 1000 to 1799, 1800 to 2499, 2500 to 3499, 3500 to 4999, 5000 to 7499, 7500 to 10000, or 10001 to 20000 micrograms/tumor/month. In specific embodiments, the dose may be at least approximately 100, 200, 400, 650, 1000, 1400, 2000, 2500, 3000, 3500, 4000, 4500, 5000, 7500, 10000, or 20000 micrograms/tumor/month.

In another embodiment, the effective dose of immunoconjugate results in an intratumoral concentration of at least approximately 5, 10, 20, 30, 40, 50, 60, 75, 100, 125, 150, 100, 200, 300, 400, or 500 micrograms/cm³ of the immunoconjugate. In other embodiments, the resulting intratumoral concentration of immunoconjugate is approximately 5 to 500, 10 to 400, 15 to 300, 20 to 200, 25 to 100, 30 to 90, 35 to 80, 40 to 70, 45 to 60, or 50 to 55 micrograms/cm³. In other embodiments, the resulting intratumoral concentration of immunoconjugate is approximately 10 to 15, 16 to 20, 21 to 25, 26 to 30, 31 to 35, 36 to 40, 41 to 45, 46 to 50, 51 to 55, 56 to 60, 61 to 65, 66 to 70, 71 to 75, 76 to 80, 81 to 85, 86 to 90, 91 to 95, 96 to 100, or 100 to 200 micrograms/cm³.

In another embodiment, the effective dose of immunoconjugate results in a plasma concentration of less than approximately 0.1, 1, 2.5, 5, 7.5, 10, 15, 20, 30, 40, or 50 micrograms/liter. In other embodiments, the resulting circulating concentration of immunoconjugate is approximately 0.1 to 50, 1 to 40, 2.5 to 30, 5 to 20, or 7.5 to 10 micrograms/liter. In other embodiments, the resulting circulating concentration of immunoconjugate is approximately 0.1 to 1, 1.1 to 2.4, 2.5 to 5, 5.1 to 7.4, 7.5 to 10, 11 to 15, 16 to 20, 21 to 30, 31 to 40, or 41 to 50 micrograms/liter.

In a particular non-limiting embodiment, the effective dose of the immunoconjugate is between about 100 and 3000 micrograms/tumor/month, for example approximately 100, 200, 300, 400, 750, or 1000 micrograms/tumor/month, wherein the patient is administered a single dose per day. The single dose is administered approximately every month for approximately 1, 2, 3, 4, 5, or 6 consecutive months. After this cycle, a subsequent cycle may begin approximately 1, 2, 4, 6 or 12 months later. The treatment regime may include 1, 2, 3, 4, 5, or 6 cycles, each cycle being spaced apart by approximately 1, 2, 4, 6, or 12 months.

In a particular non-limiting embodiment, the effective dose of the immunoconjugate is between about 20 and 1240 micrograms/tumor/day, for example approximately 20, 40, 80, 130, 200, or 280 micrograms/tumor/day or approximately 100, 200, 330, 500, 700, 930, 1240 micrograms/tumor/day, wherein the patient is administered a single dose per day. The single dose is administered approximately every day (one or more days may optionally be skipped) for approximately 1, 2, 3, 4, 5, 6 or 7 consecutive days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks.

In one embodiment, the effective dose by direct administration of the VB4-845 immunoconjugate at the cancer site may range from about 100 micrograms/day to about 2500 micrograms/day, about 200 micrograms/day to about 2500 micrograms/day, about 300 micrograms/day to about 2500 micrograms/day, about 400 micrograms/day to about 2500 micrograms/day, about 500 micrograms/day to about 2500 micrograms/day, about 600 micrograms/day to about 2500 micrograms/day, about 700 micrograms/day to about 2500 micrograms/day, about 800 micrograms/day to about 2500 micrograms/day, about 900 micrograms/day to about 2500 micrograms/day, about 1000 micrograms/day to about 2500 micrograms/day, about 1100 micrograms/day to about 2500 micrograms/day, about 1200 micrograms/day to about 2500 micrograms/day, about 1300 micrograms/day to about 2500 micrograms/day, about 1400 micrograms/day to about 2500 micrograms/day, about 1500 micrograms/day to about 2500 micrograms/day, or about 2000 micrograms/day to about 2500 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 100 micrograms/day to about 2500 micrograms/day, about 100 micrograms/day to about 2400 micrograms/day, about 100 micrograms/day to about 2300 micrograms/day, about 100 micrograms/day to about 2200 micrograms/day, about 100 micrograms/day to about 2100 micrograms/day, about 100 micrograms/day to about 2000 micrograms/day, about 100 micrograms/day to about 1900 micrograms/day, about 100 micrograms/day to about 1800 micrograms/day, about 100 micrograms/day to about 1700 micrograms/day, about 100 micrograms/day to about 1600 micrograms/day, about 100 micrograms/day to about 1500 micrograms/day, about 100 micrograms/day to about 1400 micrograms/day, about 100 micrograms/day to about 1300 micrograms/day, about 100 micrograms/day to about 1200 micrograms/day, about 100 micrograms/day to about 1100 micrograms/day, about 100 micrograms/day to about 1000 micrograms/day, about 100 micrograms/day to about 900 micrograms/day, about 100 micrograms/day to about 800 micrograms/day, about 100 micrograms/day to about 700 micrograms/day, about 100 micrograms/day to about 600 micrograms/day, about 100 micrograms/day to about 500 micrograms/day, about 100 micrograms/day to about 400 micrograms/day, about 100 micrograms/day to about 300 micrograms/day, or about 100 micrograms/day to about 200 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 200 micrograms/day to about 2500 micrograms/day, about 200 micrograms/day to about 2400 micrograms/day, about 200 micrograms/day to about 2300 micrograms/day, about 200 micrograms/day to about 2200 micrograms/day, about 200 micrograms/day to about 2100 micrograms/day, about 200 micrograms/day to about 2000 micrograms/day, about 200 micrograms/day to about 1900 micrograms/day, about 200 micrograms/day to about 1800 micrograms/day, about 200 micrograms/day to about 1700 micrograms/day, about 200 micrograms/day to about 1600 micrograms/day, about 200 micrograms/day to about 1500 micrograms/day, about 200 micrograms/day to about 1400 micrograms/day, about 200 micrograms/day to about 1300 micrograms/day, about 200 micrograms/day to about 1200 micrograms/day, about 200 micrograms/day to about 1100 micrograms/day, about 200 micrograms/day to about 1000 micrograms/day, about 200 micrograms/day to about 900 micrograms/day, about 200 micrograms/day to about 800 micrograms/day, about 200 micrograms/day to about 700 micrograms/day, about 200 micrograms/day to about 600 micrograms/day, about 200 micrograms/day to about 500 micrograms/day, about 200 micrograms/day to about 400 micrograms/day, or about 200 micrograms/day to about 300 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 300 micrograms/day to about 2500 micrograms/day, about 300 micrograms/day to about 2400 micrograms/day, about 300 micrograms/day to about 2300 micrograms/day, about 300 micrograms/day to about 2200 micrograms/day, about 300 micrograms/day to about 2100 micrograms/day, about 300 micrograms/day to about 2000 micrograms/day, about 300 micrograms/day to about 1900 micrograms/day, about 300 micrograms/day to about 1800 micrograms/day, about 300 micrograms/day to about 1700 micrograms/day, about 300 micrograms/day to about 1600 micrograms/day, about 300 micrograms/day to about 1500 micrograms/day, about 300 micrograms/day to about 1400 micrograms/day, about 300 micrograms/day to about 1300 micrograms/day, about 300 micrograms/day to about 1200 micrograms/day, about 300 micrograms/day to about 1100 micrograms/day, about 300 micrograms/day to about 1000 micrograms/day, about 300 micrograms/day to about 900 micrograms/day, about 300 micrograms/day to about 800 micrograms/day, about 300 micrograms/day to about 700 micrograms/day, about 300 micrograms/day to about 600 micrograms/day, about 300 micrograms/day to about 500 micrograms/day, or about 300 micrograms/day to about 400 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 400 micrograms/day to about 2500 micrograms/day, about 400 micrograms/day to about 2400 micrograms/day, about 400 micrograms/day to about 2300 micrograms/day, about 400 micrograms/day to about 2200 micrograms/day, about 400 micrograms/day to about 2100 micrograms/day, about 400 micrograms/day to about 2000 micrograms/day, about 400 micrograms/day to about 1900 micrograms/day, about 400 micrograms/day to about 1800 micrograms/day, about 400 micrograms/day to about 1700 micrograms/day, about 400 micrograms/day to about 1600 micrograms/day, about 400 micrograms/day to about 1500 micrograms/day, about 400 micrograms/day to about 1400 micrograms/day, about 400 micrograms/day to about 1300 micrograms/day, about 400 micrograms/day to about 1200 micrograms/day, about 400 micrograms/day to about 1100 micrograms/day, about 400 micrograms/day to about 1000 micrograms/day, about 400 micrograms/day to about 900 micrograms/day, about 400 micrograms/day to about 800 micrograms/day, about 400 micrograms/day to about 700 micrograms/day, about 400 micrograms/day to about 600 micrograms/day, or about 400 micrograms/day to about 500 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 500 micrograms/day to about 2500 micrograms/day, about 500 micrograms/day to about 2400 micrograms/day, about 500 micrograms/day to about 2300 micrograms/day, about 500 micrograms/day to about 2200 micrograms/day, about 500 micrograms/day to about 2100 micrograms/day, about 500 micrograms/day to about 2000 micrograms/day, about 500 micrograms/day to about 1900 micrograms/day, about 500 micrograms/day to about 1800 micrograms/day, about 500 micrograms/day to about 1700 micrograms/day, about 500 micrograms/day to about 1600 micrograms/day, about 500 micrograms/day to about 1500 micrograms/day, about 500 micrograms/day to about 1400 micrograms/day, about 500 micrograms/day to about 1300 micrograms/day, about 500 micrograms/day to about 1200 micrograms/day, about 500 micrograms/day to about 1100 micrograms/day, about 500 micrograms/day to about 1000 micrograms/day, about 500 micrograms/day to about 900 micrograms/day, about 500 micrograms/day to about 800 micrograms/day, about 500 micrograms/day to about 700 micrograms/day, or about 500 micrograms/day to about 600 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 600 micrograms/day to about 2500 micrograms/day, about 600 micrograms/day to about 2400 micrograms/day, about 600 micrograms/day to about 2300 micrograms/day, about 600 micrograms/day to about 2200 micrograms/day, about 600 micrograms/day to about 2100 micrograms/day, about 600 micrograms/day to about 2000 micrograms/day, about 600 micrograms/day to about 1900 micrograms/day, about 600 micrograms/day to about 1800 micrograms/day, about 600 micrograms/day to about 1700 micrograms/day, about 600 micrograms/day to about 1600 micrograms/day, about 600 micrograms/day to about 1500 micrograms/day, about 600 micrograms/day to about 1400 micrograms/day, about 600 micrograms/day to about 1300 micrograms/day, about 600 micrograms/day to about 1200 micrograms/day, about 600 micrograms/day to about 1100 micrograms/day, about 600 micrograms/day to about 1000 micrograms/day, about 600 micrograms/day to about 900 micrograms/day, about 600 micrograms/day to about 800 micrograms/day, or about 600 micrograms/day to about 700 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 700 micrograms/day to about 2500 micrograms/day, about 700 micrograms/day to about 2400 micrograms/day, about 700 micrograms/day to about 2300 micrograms/day, about 700 micrograms/day to about 2200 micrograms/day, about 700 micrograms/day to about 2100 micrograms/day, about 700 micrograms/day to about 2000 micrograms/day, about 700 micrograms/day to about 1900 micrograms/day, about 700 micrograms/day to about 1800 micrograms/day, about 700 micrograms/day to about 1700 micrograms/day, about 700 micrograms/day to about 1600 micrograms/day, about 700 micrograms/day to about 1500 micrograms/day, about 700 micrograms/day to about 1400 micrograms/day, about 700 micrograms/day to about 1300 micrograms/day, about 700 micrograms/day to about 1200 micrograms/day, about 700 micrograms/day to about 1100 micrograms/day, about 700 micrograms/day to about 1000 micrograms/day, about 700 micrograms/day to about 900 micrograms/day, or about 700 micrograms/day to about 800 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In one embodiment, the effective dose by direct administration of the VB4-845 immunoconjugate at the cancer site may range from about 800 micrograms/day to about 2500 micrograms/day, about 800 micrograms/day to about 2400 micrograms/day, about 800 micrograms/day to about 2300 micrograms/day, about 800 micrograms/day to about 2200 micrograms/day, about 800 micrograms/day to about 2100 micrograms/day, about 800 micrograms/day to about 2000 micrograms/day, about 800 micrograms/day to about 1900 micrograms/day, about 800 micrograms/day to about 1800 micrograms/day, about 800 micrograms/day to about 1700 micrograms/day, about 800 micrograms/day to about 1600 micrograms/day, about 800 micrograms/day to about 1500 micrograms/day, about 800 micrograms/day to about 1400 micrograms/day, about 800 micrograms/day to about 1300 micrograms/day, about 800 micrograms/day to about 1200 micrograms/day, about 800 micrograms/day to about 1100 micrograms/day, about 800 micrograms/day to about 1000 micrograms/day, or about 800 micrograms/day to about 900 micrograms/day. The dosage may be administered everyday for 1 day, 2 days, 3 days, 4 days, 5 days, 6 days, 7 days, 8 days, 9 days, 10 days, 11 days, 12 days, 13 days, 14 days, 15 days, 21 days, 28 days, 35 days, 42 days, 49 days, 56 days, 63 days, or 70 days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. Stock VB4-845 may be diluted with phosphate buffered saline or any other sterile solutions to obtain the required concentration for administration.

In some embodiments, the effective dose by direct administration of the VB4-845 immunoconjugate at the cancer site may range from about 100 micrograms/week to about 5000 micrograms/week, about 100 micrograms/week to about 4500 micrograms/week, about 100 micrograms/week to about 4000 micrograms/week, about 100 micrograms/week to about 3500 micrograms/week, about 100 micrograms/week to about 3000 micrograms/week, about 100 micrograms/week to about 2500 micrograms/week, about 100 micrograms/week to about 2000 micrograms/week, about 100 micrograms/week to about 1800 micrograms/week, about 100 micrograms/week to about 1700 micrograms/week, about 100 micrograms/week to about 1600 micrograms/week, about 100 micrograms/week to about 1500 micrograms/week, about 100 micrograms/week to about 1400 micrograms/week, about 100 micrograms/week to about 1300 micrograms/week, about 100 micrograms/week to about 1200 micrograms/week, about 100 micrograms/week to about 1100 micrograms/week, about 100 micrograms/week to about 1000 micrograms/week, about 100 micrograms/week to about 900 micrograms/week, about 100 micrograms/week to about 800 micrograms/week, about 100 micrograms/week to about 700 micrograms/week, about 100 micrograms/week to about 600 micrograms/week, about 100 micrograms/week to about 500 micrograms/week, about 100 micrograms/week to about 400 micrograms/week, about 100 micrograms/week to about 300 micrograms/week, or about 100 micrograms/week to about 200 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 200 micrograms/week to about 5000 micrograms/week, about 200 micrograms/week to about 4500 micrograms/week, about 200 micrograms/week to about 4000 micrograms/week, about 200 micrograms/week to about 3500 micrograms/week, about 200 micrograms/week to about 3000 micrograms/week, about 200 micrograms/week to about 2500 micrograms/week, about 200 micrograms/week to about 2000 micrograms/week, about 200 micrograms/week to about 1800 micrograms/week, about 200 micrograms/week to about 1700 micrograms/week, about 200 micrograms/week to about 1600 micrograms/week, about 200 micrograms/week to about 1500 micrograms/week, about 200 micrograms/week to about 1400 micrograms/week, about 200 micrograms/week to about 1300 micrograms/week, about 200 micrograms/week to about 1200 micrograms/week, about 200 micrograms/week to about 1100 micrograms/week, about 200 micrograms/week to about 1000 micrograms/week, about 200 micrograms/week to about 900 micrograms/week, about 200 micrograms/week to about 800 micrograms/week, about 200 micrograms/week to about 700 micrograms/week, about 200 micrograms/week to about 600 micrograms/week, about 200 micrograms/week to about 500 micrograms/week, about 200 micrograms/week to about 400 micrograms/week, or about 200 micrograms/week to about 300 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 300 micrograms/week to about 5000 micrograms/week, about 300 micrograms/week to about 4500 micrograms/week, about 300 micrograms/week to about 4000 micrograms/week, about 300 micrograms/week to about 3500 micrograms/week, about 300 micrograms/week to about 3000 micrograms/week, about 300 micrograms/week to about 2500 micrograms/week, about 300 micrograms/week to about 2000 micrograms/week, about 300 micrograms/week to about 1800 micrograms/week, about 300 micrograms/week to about 1700 micrograms/week, about 300 micrograms/week to about 1600 micrograms/week, about 300 micrograms/week to about 1500 micrograms/week, about 300 micrograms/week to about 1400 micrograms/week, about 300 micrograms/week to about 1300 micrograms/week, about 300 micrograms/week to about 1200 micrograms/week, about 300 micrograms/week to about 1100 micrograms/week, about 300 micrograms/week to about 1000 micrograms/week, about 300 micrograms/week to about 900 micrograms/week, about 300 micrograms/week to about 800 micrograms/week, about 300 micrograms/week to about 700 micrograms/week, about 300 micrograms/week to about 600 micrograms/week, about 300 micrograms/week to about 500 micrograms/week, or about 300 micrograms/week to about 400 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 400 micrograms/week to about 5000 micrograms/week, about 400 micrograms/week to about 4500 micrograms/week, about 400 micrograms/week to about 4000 micrograms/week, about 400 micrograms/week to about 3500 micrograms/week, about 400 micrograms/week to about 3000 micrograms/week, about 400 micrograms/week to about 2500 micrograms/week, about 400 micrograms/week to about 2000 micrograms/week, about 400 micrograms/week to about 1800 micrograms/week, about 400 micrograms/week to about 1700 micrograms/week, about 400 micrograms/week to about 1600 micrograms/week, about 400 micrograms/week to about 1500 micrograms/week, about 400 micrograms/week to about 1400 micrograms/week, about 400 micrograms/week to about 1300 micrograms/week, about 400 micrograms/week to about 1200 micrograms/week, about 400 micrograms/week to about 1100 micrograms/week, about 400 micrograms/week to about 1000 micrograms/week, about 400 micrograms/week to about 900 micrograms/week, about 400 micrograms/week to about 800 micrograms/week, about 400 micrograms/week to about 700 micrograms/week, about 400 micrograms/week to about 600 micrograms/week, or about 400 micrograms/week to about 500 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 500 micrograms/week to about 5000 micrograms/week, about 500 micrograms/week to about 4500 micrograms/week, about 500 micrograms/week to about 4000 micrograms/week, about 500 micrograms/week to about 3500 micrograms/week, about 500 micrograms/week to about 3000 micrograms/week, about 500 micrograms/week to about 2500 micrograms/week, about 500 micrograms/week to about 2000 micrograms/week, about 500 micrograms/week to about 1800 micrograms/week, about 500 micrograms/week to about 1700 micrograms/week, about 500 micrograms/week to about 1600 micrograms/week, about 500 micrograms/week to about 1500 micrograms/week, about 500 micrograms/week to about 1400 micrograms/week, about 500 micrograms/week to about 1300 micrograms/week, about 500 micrograms/week to about 1200 micrograms/week, about 500 micrograms/week to about 1100 micrograms/week, about 500 micrograms/week to about 1000 micrograms/week, about 500 micrograms/week to about 900 micrograms/week, about 500 micrograms/week to about 800 micrograms/week, about 500 micrograms/week to about 700 micrograms/week, or about 500 micrograms/week to about 600 micrograms/week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 600 micrograms/week to about 5000 micrograms/week, about 600 micrograms/week to about 4500 micrograms/week, about 600 micrograms/week to about 4000 micrograms/week, about 600 micrograms/week to about 3500 micrograms/week, about 600 micrograms/week to about 3000 micrograms/week, about 600 micrograms/week to about 2500 micrograms/week, about 600 micrograms/week to about 2000 micrograms/week, about 600 micrograms/week to about 1800 micrograms/week, about 600 micrograms/week to about 1700 micrograms/week, about 600 micrograms/week to about 1600 micrograms/week, about 600 micrograms/week to about 1500 micrograms/week, about 600 micrograms/week to about 1400 micrograms/week, about 600 micrograms/week to about 1300 micrograms/week, about 600 micrograms/week to about 1200 micrograms/week, about 600 micrograms/week to about 1100 micrograms/week, about 600 micrograms/week to about 1000 micrograms/week, about 600 micrograms/week to about 900 micrograms/week, about 600 micrograms/week to about 800 micrograms/week, or about 600 micrograms/week to about 700 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 700 micrograms/week to about 5000 micrograms/week, about 700 micrograms/week to about 4500 micrograms/week, about 700 micrograms/week to about 4000 micrograms/week, about 700 micrograms/week to about 3500 micrograms/week, about 700 micrograms/week to about 3000 micrograms/week, about 700 micrograms/week to about 2500 micrograms/week, about 700 micrograms/week to about 2000 micrograms/week, about 700 micrograms/week to about 1800 micrograms/week, about 700 micrograms/week to about 1700 micrograms/week, about 700 micrograms/week to about 1600 micrograms/week, about 700 micrograms/week to about 1500 micrograms/week, about 700 micrograms/week to about 1400 micrograms/week, about 700 micrograms/week to about 1300 micrograms/week, about 700 micrograms/week to about 1200 micrograms/week, about 700 micrograms/week to about 1100 micrograms/week, about 700 micrograms/week to about 1000 micrograms/week, about 700 micrograms/week to about 900 micrograms/week, or about 700 micrograms/week to about 800 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 800 micrograms/week to about 5000 micrograms/week, about 800 micrograms/week to about 4500 micrograms/week, about 800 micrograms/week to about 4000 micrograms/week, about 800 micrograms/week to about 3500 micrograms/week, about 800 micrograms/week to about 3000 micrograms/week, about 800 micrograms/week to about 2500 micrograms/week, about 800 micrograms/week to about 2000 micrograms/week, about 800 micrograms/week to about 1800 micrograms/week, about 800 micrograms/week to about 1700 micrograms/week, about 800 micrograms/week to about 1600 micrograms/week, about 800 micrograms/week to about 1500 micrograms/week, about 800 micrograms/week to about 1400 micrograms/week, about 800 micrograms/week to about 1300 micrograms/week, about 800 micrograms/week to about 1200 micrograms/week, about 800 micrograms/week to about 1100 micrograms/week, about 800 micrograms/week to about 1000 micrograms/week, or about 800 micrograms/week to about 900 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 900 micrograms/week to about 5000 micrograms/week, about 900 micrograms/week to about 4500 micrograms/week, about 900 micrograms/week to about 4000 micrograms/week, about 900 micrograms/week to about 3500 micrograms/week, about 900 micrograms/week to about 3000 micrograms/week, about 900 micrograms/week to about 2500 micrograms/week, about 900 micrograms/week to about 2000 micrograms/week, about 900 micrograms/week to about 1800 micrograms/week, about 900 micrograms/week to about 1700 micrograms/week, about 900 micrograms/week to about 1600 micrograms/week, about 900 micrograms/week to about 1500 micrograms/week, about 900 micrograms/week to about 1400 micrograms/week, about 900 micrograms/week to about 1300 micrograms/week, about 900 micrograms/week to about 1200 micrograms/week, about 900 micrograms/week to about 1100 micrograms/week, or about 900 micrograms/week to about 1000 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the effective dose by direct administration of the VB4-845 immunoconjugate at the cancer site may range from about 1000 micrograms/week to about 5000 micrograms/week, about 1000 micrograms/week to about 4500 micrograms/week, about 1000 micrograms/week to about 4000 micrograms/week, about 1000 micrograms/week to about 3500 micrograms/week, about 1000 micrograms/week to about 3000 micrograms/week, about 1000 micrograms/week to about 2500 micrograms/week, about 1000 micrograms/week to about 2000 micrograms/week, about 1000 micrograms/week to about 1800 micrograms/week, about 1000 micrograms/week to about 1700 micrograms/week, about 1000 micrograms/week to about 1600 micrograms/week, about 1000 micrograms/week to about 1500 micrograms/week, about 1000 micrograms/week to about 1400 micrograms/week, about 1000 micrograms/week to about 1300 micrograms/week, about 1000 micrograms/week to about 1200 micrograms/week, or about 1000 micrograms/week to about 1100 micrograms/week. In some embodiments, a single dose may be administered in a week. In some embodiments, multiple doses may be administered in a week, for example, 2 doses, 3 doses, 4 doses, or 5 doses. A dosing cycle may include administration for 1 week, for 2 weeks, for 3 weeks, for 4 weeks, for 5 weeks, for 6 weeks, for 7 weeks, for 8 weeks, for 9 weeks, or for 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

The injection volume preferably is at least an effective amount, which is appropriate to the type and/or location of the tumor. The maximum injection volume in a single dose may be between about 25% and 75% of tumor volume, for example approximately one-quarter, one-third, or three-quarters of the estimated target tumor volume. In a specific, non-limiting embodiment, the maximum injection volume in a single dose is approximately 30% of the tumor volume.

In another embodiment, the immunoconjugate is administered intratumourally at a total dose per cycle equivalent to, or below the maximum tolerated dose established in a safety trial but the dosage is standardized in relation to the tumor volume. For example, subjects will receive between 1 microgram per cm³ and 500 microgram per cm³ tumor or a dose sufficient to reach about between 14 picomole and 7 nanomole per cm³ tumor tissue. The dose will be administered in a volume not exceeding about 20-50% of the tumor volume. The immunoconjugate will be diluted in a suitable salt solution. For example, for a tumor of estimated volume of 3 cm³, a target dose of 14 picomoles (1 microgram per cm³), and a maximum injection relative volume of about ⅓ of the tumor, 3 microgram of immunoconjugate will be diluted into about 1 ml of diluent.

In another particular embodiment, the effective dose of the immunoconjugate is between about 20 and 300 micro-grams/tumor/day, for example approximately 20, 40, 80, 130, 200, or 280 micrograms/tumor/day, wherein the patient is administered a single dose per day. The maximum injection volume in a single dose may be between about 25% and 75% of tumor volume, for example approximately one-quarter, one-third, or three-quarters of the estimated target tumor volume. The single dose is administered every other day for approximately 5, 7, 9, 11, 13, 15, 17, 19, 21, 23, 25, 27, 29, or 31 consecutive days. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In one specific non-limiting embodiment, an immuconjugate, such as VB4-845 is administered at a dose of approximately 280 micrograms/tumor/day, wherein the patient is administered a single dose per day. The maximum injection volume in a single dose is approximately one-third of the estimated target tumor volume. The single dose is administered every day for approximately five consecutive days. After this cycle, a subsequent cycle may begin approximately one month later, preferably one month from the first day of the first cycle. The treatment regime may include three cycles, each cycle being spaced apart by approximately one treatment-free week.

In another specific non-limiting embodiment, an immuconjugate, such as VB4-845 is administered at a dose of approximately 280 micrograms/tumor/day, wherein the patient is administered a single dose per day. The maximum injection volume in a single dose is approximately one-third of the estimated target tumor volume. The single dose is administered every other day for approximately one week. After this cycle, a subsequent cycle may begin approximately one week later. The treatment regime may include three cycles, each cycle being spaced apart by approximately one week.

In yet another specific embodiment, an immuconjugate, such as VB4-845 is administered at a dose of approximately 280 micrograms/tumor/day, wherein the patient is administered a single dose per day. The maximum injection volume in a single dose is approximately one-third of the estimated target tumor volume. The single dose is administered every other day for approximately three weeks. After this cycle, a subsequent cycle may begin approximately one week later. The treatment regime may include three cycles, each cycle being spaced apart by approximately one week.

For administration to a cavity such as peritoneal cavity, the effective dose of the immunoconjugate is between about 100 and 2000 micrograms in 50 ml/week, for example approximately 100, 200, 335, 500, 700, 930, 1240 micrograms in 50 ml/week, wherein the patient is administered a single dose per week and the tumor tissue is exposed to the immunoconjugate for at least about 30 minutes. For example, the solution is retained into the cavity for about 30 minutes to about 3 hours. In a specific non-limiting embodiment, the tumor tissue is exposed to the immunoconjugate for about 1 hours or more preferably for about 2 hours. After this cycle, a subsequent cycle may begin approximately 1, 2, 4, 6, or 12 weeks after the previous dose. The treatment regime may include 1, 2, 3, 4, 5, or 6 cycles, each cycle being spaced apart by approximately 1, 2, 4, 6, or 12 months.

Dosage for the immunoconjugate can also be expressed as molarity of the binding site for the protein on the cancer cells. For example, the immunoconjugate VB4-845 has a molecular weight of about 69.7 kDa and one binding site for Ep-CAM. It is known that other immunoconjugate formats such as divalent formats, Fab, Fabl′ or (Fabl′)₂ fragment could have a different molecular weight by virtue of the number of amino acids in the polypeptide chain or chains. It is also known that for a similar format one could alter the molecular weight by attaching additional groups to the polypeptide such sugar moiety or polyethylene glycol. The use of a different toxin or a different variant of the toxin could also result in an immunoconjugate with a different molecular weight than VB4-845 used in the examples. Furthermore, changes to the polypeptide chain that result in a longer or a shorter fragment could also be made and yet without losing the binding of the immunoconjugate to the chosen protein on the cancer cell. All those variations are contemplated in this application. As a result it may be helpful to express the dosage of the immunoconjugate in terms of the number of moles of the binding sites for the protein on the cancer cells. In the examples and the various embodiments, the dosages are expressed in micrograms and are based on the molecular weight of VB4-845.

The effective dose of another anticancer agent to be administered together with an immunoconjugate during a cycle also varies according to the mode of administration. The one or more anticancer agent may be delivered intratumorally, or by other modes of administration. Typically, chemotherapeutic agents are administered systemically. Standard dosage and treatment regimens are known in the art (see, e.g., the latest editions of the Merck Index and the Physician's Desk Reference).

For example, in one embodiment, the additional anticancer agent comprises dacarbazine at a dose ranging from approximately 200 to 4000 mg/m²/cycle. In a preferred embodiment, the dose ranges from 700 to 1000 mg/m²/cycle.

In another embodiment, the additional anticancer agent comprises fludarabine at a dose ranging from approximately 25 to 50 mg/m²/cycle.

In another embodiment, the additional anticancer agent comprises cytosine arabinoside (Ara-C) at a dose ranging from approximately 200 to 2000 mg/m²/cycle.

In another embodiment, the additional anticancer agent comprises docetaxel at a dose ranging from approximately 1.5 to 7.5 mg/kg/cycle.

In another embodiment, the additional anticancer agent comprises paclitaxel at a dose ranging from approximately 5 to 15 mg/kg/cycle.

In yet another embodiment, the additional anticancer agent comprises cisplatin at a dose ranging from approximately 5 to 20 mg/kg/cycle.

In yet another embodiment, the additional anticancer agent comprises 5-fluorouracil at a dose ranging from approximately 2 mg/kg to about 20 mg/kg, 2 mg/kg to about 18 mg/kg, 2 mg/kg to about 16 mg/kg, 2 mg/kg to about 14 mg/kg, 2 mg/kg to about 12 mg/kg, 2 mg/kg to about 10 mg/kg, 2 mg/kg to about 8 mg/kg, 2 mg/kg to about 6 mg/kg, or 2 mg/kg to about 4 mg/kg. The anticancer agent may be administered daily for 1, 2, 3, 4, 5, 6, 7, 8, 9, 10, 11, 12, 13, 14, 15, 16, 17, 18, 19, 20, 21, or 30 consecutive days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks. In some embodiments, the 5-FU may be on maintainance therapy, repeating the dosage of first cycle every 30 days after the last day of the previous course of treatment.

In yet another embodiment, the additional anticancer agent comprises doxorubicin at a dose ranging from approximately 2 to 8 mg/kg/cycle.

In yet another embodiment, the additional anticancer agent comprises epipodophyllotoxin at a dose ranging from approximately 40 to 160 mg/kg/cycle.

In yet another embodiment, the additional anticancer agent comprises cyclophosphamide at a dose ranging from approximately 50 to 200 mg/kg/cycle.

In yet another embodiment, the additional anticancer agent comprises irinotecan at a dose ranging from approximately 50 to 75, 75 to 100, 100 to 125, or 125 to 150 mg/m²/cycle.

In yet another embodiment, the anticancer agent comprises vinblastine at a dose ranging from approximately 3.7 to 5.4, 5.5 to 7.4, 7.5 to 11, or 11 to 18.5 mg/m²/cycle.

In yet another embodiment, the additional anticancer agent comprises vincristine at a dose ranging from approximately 0.7 to 1.4, or 1.5 to 2 mg/m²/cycle.

In yet another embodiment, the additional anticancer agent comprises methotrexate at a dose ranging from approximately 3.3 to 5, 5 to 10, 10 to 100, or 100 to 1000 mg/m²/cycle.

In the foregoing embodiments, the anticancer agent may be administered daily for 1, 2, 3, 4, 5, 6, 7, 8, 9, 10, 11, 12, 13, 14, 15, 16, 17, 18, 19, 20, or 21 consecutive days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks.

Combination therapy with an immunoconjugate may sensitize the cancer or tumor to administration of an additional anticancer agent. Accordingly, the present invention contemplates combination therapies for preventing, treating, and/or preventing recurrence of cancer comprising administering an effective amount of an immunoconjugate prior to, subsequently, or concurrently with a reduced dose of an anticancer agent. For example, initial treatment with an immunoconjugate may increase the sensitivity of a cancer or tumor to subsequent challenge with a dose of anticancer agent. This dose is near, or below, the low range of standard dosages when the anticancer agent is administered alone, or in the absence of an immunoconjugate. When concurrently administered, the immunoconjugate may be administered separately from the anticancer agent, and optionally, via a different mode of administration.

In some embodiments, the following VB4-845 and 5-fluorouracil (5-FU) combinations may be administered:

| VB4-845 | 5-FU |
|---|---|
| 2500 μg/day | 20 mg/kg/day |
| 2500 μg/day | 15 mg/kg/day |
| 2500 μg/day | 10 mg/kg/day |
| 2500 μg/day | 6 mg/kg/day |
| 2500 μg/day | 4 mg/kg/day |
| 2500 μg/day | 2 mg/kg/day |

The VB4-845 and 5-FU combination may be administered daily for 1, 2, 3, 4, 5, 6, 7, 8, 9, 10, 11, 12, 13, 14, 15, 16, 17, 18, 19, 20, or 21 consecutive days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks.

In some embodiments, the following VB4-845 and 5-fluorouracil (5-FU) combinations may be administered:

| VB4-845 | 5-FU |
|---|---|
| 2500 μg/day | 20 mg/kg/day |
| 2000 μg/day | 20 mg/kg/day |
| 1500 μg/day | 20 mg/kg/day |
| 1000 μg/day | 20 mg/kg/day |
| 500 μg/day | 20 mg/kg/day |
| 250 μg/day | 20 mg/kg/day |

The VB4-845 and 5-FU combination may be administered daily for 1, 2, 3, 4, 5, 6, 7, 8, 9, 10, 11, 12, 13, 14, 15, 16, 17, 18, 19, 20, or 21 consecutive days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks.

In some embodiments, the following VB4-845 and 5-fluorouracil (5-FU) combinations may be administered:

| VB4-845 | 5-FU |
|---|---|
| 100-2500 μg/day | 20 mg/kg/day |
| 100-2500 μg/day | 15 mg/kg/day |
| 100-2500 μg/day | 10 mg/kg/day |
| 100-2500 μg/day | 6 mg/kg/day |
| 100-2500 μg/day | 4 mg/kg/day |
| 100-2500 μg/day | 2 mg/kg/day |

The VB4-845 and 5-FU combination may be administered daily for 1, 2, 3, 4, 5, 6, 7, 8, 9, 10, 11, 12, 13, 14, 15, 16, 17, 18, 19, 20, or 21 consecutive days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks.

Accordingly, in one embodiment, the additional anticancer agent comprises cisplatin, e.g., PLATINOL or PLATINOL-AQ (Bristol Myers), at a dose ranging from approximately 5 to 10, 11 to 20, 21 to 40, or 41 to 75 mg/m²/cycle.

In another embodiment, the additional anticancer agent comprises carboplatin, e.g., PARAPLATIN (Bristol Myers), at a dose ranging from approximately 2 to 3, 4 to 8, 9 to 16, 17 to 35, or 36 to 75 mg/m²/cycle.

In another embodiment, the additional anticancer agent comprises cyclophosphamide, e.g., CYTOXAN (Bristol Myers Squibb), at a dose ranging from approximately 0.25 to 0.5, 0.6 to 0.9, 1 to 2, 3 to 5, 6 to 10, 11 to 20, or 21 to 40 mg/kg/cycle.

In another embodiment, the additional anticancer agent comprises cytarabine, e.g., CYTOSAR-U (Pharmacia & Upjohn), at a dose ranging from approximately 0.5 to 1, 2 to 4, 5 to 10, 11 to 25, 26 to 50, or 51 to 100 mg/m²/cycle. In another embodiment, the additional anticancer agent comprises cytarabine liposome, e.g., DEPOCYT (Chiron Corp.), at a dose ranging from approximately 5 to 50 mg/m²/cycle.

In another embodiment, the additional anticancer agent comprises dacarbazine, e.g., DTIC or DTICDOME (Bayer Corp.), at a dose ranging from approximately 15 to 250 mg/m2/cycle or ranging from approximately 0.2 to 2 mg/kg/cycle.

In another embodiment, the additional anticancer agent comprises topotecan, e.g., HYCAMTIN (SmithKline Beecham), at a dose ranging from approximately 0.1 to 0.2, 0.3 to 0.4, 0.5 to 0.8, or 0.9 to 1.5 mg/m²/Cycle.

In another embodiment, the additional anticancer agent comprises irinotecan, e.g., CAMPTOSAR (Pharmacia & Upjohn), at a dose ranging from approximately 5 to 9, 10 to 25, or 26 to 50 mg/m²/cycle.

In another embodiment, the additional anticancer agent comprises fludarabine, e.g., FLUDARA (Berlex Laboratories), at a dose ranging from approximately 2.5 to 5, 6 to 10, 11 to 15, or 16 to 25 mg/m²/cycle.

In another embodiment, the additional anticancer agent comprises cytosine arabinoside (Ara-C) at a dose ranging from approximately 200 to 2000 mg/m2/cycle, 300 to 1000 mg/m2/cycle, 400 to 800 mg/m²/cycle, or 500 to 700 mg/m²/cycle.

In another embodiment, the additional anticancer agent comprises docetaxel, e.g., TAXOTERE (Rhone Poulenc Rorer) at a dose ranging from approximately 6 to 10, 11 to 30, or 31 to 60 mg/m²/cycle.

In another embodiment, the additional anticancer agent comprises paclitaxel, e.g., TAXOL (Bristol Myers Squibb), at a dose ranging from approximately 10 to 20, 21 to 40, 41 to 70, or 71 to 135 mg/kg/cycle.

In another embodiment, the additional anticancer agent comprises 5-fluorouracil at a dose ranging from approximately 0.5 to 5 mg/kg/cycle, 1 to 4 mg/kg/cycle, or 2-3 mg/kg/cycle.

In another embodiment, the additional anticancer agent comprises doxorubicin, e.g., ADRIAMYCIN (Pharmacia & Upjohn), DOXIL (Alza), RUBEX (Bristol Myers Squibb), at a dose ranging from approximately 2 to 4, 5 to 8, 9 to 15, 16 to 30, or 31 to 60 mg/kg/cycle.

In another embodiment, the additional anticancer agent comprises etoposide, e.g., VEPESID (Pharmacia & Upjohn), at a dose ranging from approximately 3.5 to 7, 8 to 15, 16 to 25, or 26 to 50 mg/m²/cycle.

In another embodiment, the additional anticancer agent comprises vinblastine, e.g., VELBAN (Eli Lilly), at a dose ranging from approximately 0.3 to 0.5, 0.6 to 0.9, 1 to 2, or 3 to 3.6 mg/m2/cycle.

In another embodiment, the additional anticancer agent comprises vincristine, e.g., ONCOVIN (Eli Lilly), at a dose ranging from approximately 0.1, 0.2, 0.3, 0.4, 0.5, 0.6 or 0.7 mg/m²/cycle.

In another embodiment, the additional anticancer agent comprises methotrexate at a dose ranging from approximately 0.2 to 0.9, 1 to 5, 6 to 10, or 11 to 20 mg/m²/cycle.

In another embodiment, an immunoconjugate is administered in combination with at least one other immunotherapeutic which includes, without limitation, rituxan, rituximab, campath-1, gemtuzumab, and trastuzutmab.

In another embodiment, an immunoconjugate is administered in combination with one or more anti-angiogenic agents which include, without limitation, angiostatin, thalidomide, kringle 5, endostatin, Serpin (Serine Protease Inhibitor), anti-thrombin, 29 kDa N-terminal and a 40 kDa C-terminal proteolytic fragments of fibronectin, 16 kDa proteolytic fragment of prolactin, 7.8 kDa proteolytic fragment of platelet factor-4, a 13 amino acid peptide corresponding to a fragment of platelet factor-4, a 14-amino acid peptide corresponding to a fragment of collagen I, a 19 amino acid peptide corresponding to a fragment of thrombospondin I, a 20-amino acid peptide corresponding to a fragment of SPARC, and a variant thereof, including a pharmaceutically acceptable salt thereof.

In another embodiment, an immunoconjugate is administered in combination with one or more cytokines which include, without limitation, a lymphokine, tumor necrosis factors, tumor necrosis factor-like cytokine, lymphotoxin, interferon, macrophage inflammatory protein, granulocyte monocyte colony stimulating factor, interleukin (including, without limitation, interleukin-1, interleukin-2, interleukin-6, interleukin-12, interleukin-15, interleukin-18), and a variant thereof, including a pharmaceutically acceptable salt thereof.

In yet another embodiment, an immunoconjugate is administered in combination with a cancer vaccine including, without limitation, autologous cells or tissues, non-autologous cells or tissues, carcinoembryonic antigen, alpha-feto-protein, human chorionic gonadotropin, BCG live vaccine, melanocyte lineage proteins, and mutated, tumor-specific antigens.

In yet another embodiment, an immunoconjugate is administered in association with hormonal therapy. Hormonal therapeutics include, without limitation, a hormonal agonist, hormonal antagonist (e.g., flutamide, tamoxifen, leuprolide acetate (LUPRON)), and steroid (e.g., dexamethasone, retinoid, betamethasone, cortisol, cortisone, prednisone, dehydrotestosterone, glucocorticoid, mineralocorticoid, estrogen, testosterone, progestin).

In yet another embodiment, an immunoconjugate is administered in association with a gene therapy program to treat or prevent cancer.

In yet another embodiment, an Ep-CAM-targeted immunoconjugate is administered in combination with one or more agents that increase expression of Ep-CAM in the tumor cells of interest. Ep-CAM expression preferably is increased so that a greater number of Ep-CAM molecules are expressed on the tumor cell surface. For example, the agent may inhibit the normal cycles of Ep-CAM antigen endocytosis. Such combination treatment may improve the clinical efficacy of the Ep-CAM-targeted immunoconjugate alone, or with other anticancer agents or radiation therapy. In specific, nonlimiting embodiments, the agent which increases Ep-CAM expression in the tumor cells is vinorelbine tartrate (Navelbine) and/or paclitaxel (Taxol).

Combination therapy may thus increase the sensitivity of the cancer or tumor to the administered immunoconjugate and/or additional anticancer agent. In this manner, shorter treatment cycles may be possible thereby reducing toxic events. Accordingly, the invention provides a method for treating or preventing cancer comprising administering to a patient in need thereof an effective amount of an immunoconjugate and at least one other anticancer agent for a short treatment cycle. The cycle duration may range from approximately 1 to 30, 2 to 27, 3 to 15, 4 to 12, 5 to 9, or 6-8 days. The cycle duration may vary according to the specific anticancer agent in use. The invention also contemplates continuous or discontinuous administration, or daily doses divided into several partial administrations. An appropriate cycle duration for a specific anticancer agent will be appreciated by the skilled artisan, and the invention contemplates the continued assessment of optimal treatment schedules for each anticancer agent. Specific guidelines for the skilled artisan are known in the art.

Alternatively, longer treatment cycles may be desired. Accordingly, the cycle duration may range from approximately 10 to 56, 12 to 48, 14 to 28, 16 to 24, or 18 to 20 days. The cycle duration may vary according to the specific anticancer agent in use.

The present invention contemplates at least one cycle, preferably more than one cycle during which a single anticancer agent or series of agents is administered. An appropriate total number of cycles, and the interval between cycles, will be appreciated by the skilled artisan. The number of cycles may be 1, 2, 3, 4, 5, 6, 7, 8, 9, 10, 11, 12, 13, 14, 15, 16, 17, 18, 19, 20, or 21 cycles. The interval between cycles may be 1, 2, 3, 4, 5, 6, 7, 8, 9, 10, 11, 12, 13, 14, 15, 16, 17, 18, 19, 20, or 21 days. The invention contemplates the continued assessment of optimal treatment schedules for each immunoconjugate and additional anticancer agent.

In one non-limiting embodiment of the invention, the immunoconjugate is directly administered at high doses (e.g., a dose resulting in greater than approximately 100, 200, 300, 400, 500, or 1000 micrograms/cm³) for shorter periods. Accordingly, in one non-limiting, specific embodiment, the immunoconjugate is administered intratumorally at a dose that results in an intratumoral concentration of immunoconjugate of at least approximately 200, 300, 400, or 500 micrograms/cm³ once a week for two weeks.

An immunoconjugate according to the invention may be comprised in a pharmaceutical composition or medicament. Pharmaceutical compositions adapted for direct administration include, without limitation, lyophilized powders or aqueous or non-aqueous sterile injectable solutions or suspensions, which may further contain antioxidants, buffers, bacteriostats and solutes that render the compositions substantially isotonic with the blood of an intended recipient. Other components that may be present in such compositions include water, alcohols, polyols, glycerin and vegetable oils, for example. Extemporaneous injection solutions and suspensions may be prepared from sterile powders, granules and tablets. Immunoconjugate may be supplied, for example but not by way of limitation, as a lyophilized powder which is reconstituted with sterile water or saline prior to administration to the patient.

Pharmaceutical compositions of the invention may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy) propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

In another embodiment, a pharmaceutical composition comprises an immunoconjugate and one or more additional anticancer agent, optionally in a pharmaceutically acceptable carrier.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

In various embodiments of the invention, the pharmaceutical composition is directly administered to the area of the tumor(s) by, for example, local infusion during surgery, topical application (e.g., in conjunction with a wound dressing after surgery), injection, means of a catheter, means of a suppository, or means of an implant. An implant can be of a porous, non-porous, or gelatinous material, including membranes, such as sialastic membranes, or fibers. Suppositories generally contain active ingredients in the range of 0.5% to 10% by weight.

In other embodiments, a controlled release system can be placed in proximity of the target tumor. For example, a micropump may deliver controlled doses directly into the area of the tumor, thereby finely regulating the timing and concentration of the pharmaceutical composition.

In accordance with one aspect of the present invention, the immunoconjugate and/or other anticancer agent is delivered to the patient by direct administration. Accordingly, the immunoconjugate and/or other anticancer agent may be administered, without limitation, by one or more direct injections into the tumor, by continuous or discontinuous perfusion into the tumor, by introduction of a reservoir of the immunoconjugate, by introduction of a slow-release apparatus into the tumor, by introduction of a slow-release formulation into the tumor, and/or by direct application onto the tumor. By the mode of administration into the tumor, introduction of the immunoconjugate and/or other anticancer agent to the area of the tumor, or into a blood vessel or lymphatic vessel that substantially directly flows into the area of the tumor, is also contemplated. In each case, the pharmaceutical composition is administered in at least an amount sufficient to achieve the endpoint, and if necessary, comprises a pharmaceutically acceptable carrier.

It is contemplated that the immunoconjugate may be administered intratumorally, whereas any other anticancer agent may be delivered to the patient by other modes of administration (e.g., intravenously). Additionally, where multiple anticancer agents are intended to be delivered to a patient, the immunoconjugate and one or more of the other anticancer agent may be delivered intratumorally, whereas other anticancer agents may be delivered by other modes of administration (e.g., intravenously and orally).

In some embodiments, the pharmaceutical carrier may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Pharmaceutical compositions of the peptides/compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The immunoconjugate of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the peptides/compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams. In some embodiments, the immunoconjugate administration may be directly to the cancer site.

For oral administration, the immunoconjugates can be formulated readily by combining these immunoconjugates with pharmaceutically acceptable carriers well known in the art. Such carriers enable the immunoconjugates of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active peptides/compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active peptides/compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the peptides/compound and a suitable powder base such as lactose or starch.

The compositions of the present invention can also be formulated in rectal compositions such as suppositories or retention enemas, e.g., containing conventional suppository bases such as cocoa butter or other glycerides.

In addition to the formulations described previously, the compositions of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the peptides/compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present invention, for example, can be applied to a plaster, or can be applied by transdermal, therapeutic systems that are consequently supplied to the organism.

The compositions of the present invention can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### EXAMPLES

### Example 1

### VB4-845 Construct

VB4-845 is an immunoconjugate comprised of a single-chain Fv recombinant human antibody fragment that is fused to a truncated form of Pseudomonas exotoxin A (ETA 252-608). The antibody fragment is derived from the humanized MOC31 single-chain antibody fragment, 4D5MOCB, which specifically binds to Ep-CAM.

Exotoxin A is one of the toxic proteins released by pathogenic strains of Pseudomonas aeruginosa. It is secreted as a proenzyme with a molecular weight of 66,000 daltons. Exotoxin A is translocated into susceptible mammalian cells, where covalent alteration of the molecule renders it enzymatically active. Pseudomonas exotoxin A irreversibly blocks protein synthesis in cells by adenosine diphosphate-ribosylating a post-translationally modified histidine residue of elongation factor-2, called diphthamide, and induces apoptosis. The truncated version of ETA used in this construct, while still containing the domains for inducing cell death, lacks the cell-binding domain, thereby preventing the ETA portion from entering cells absent targeting by the antibody portion of the immunoconjugate.

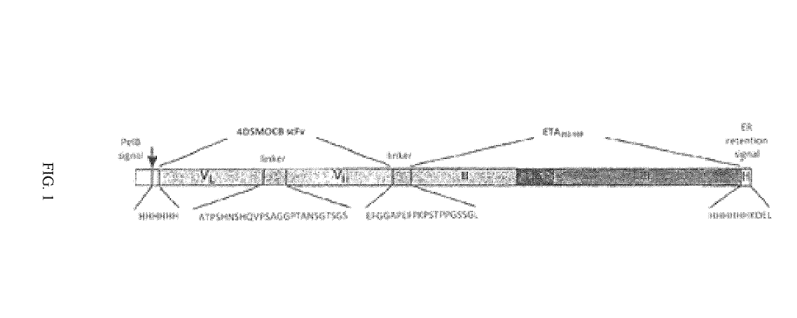

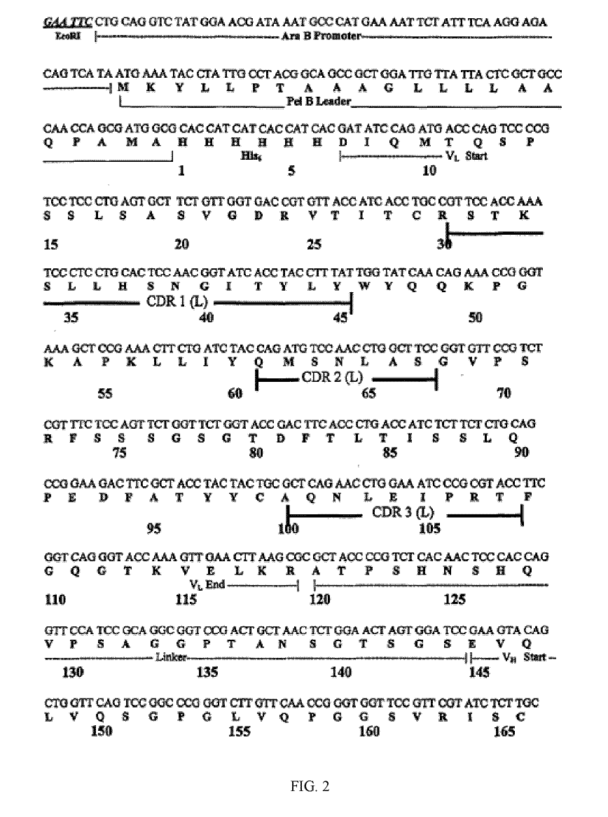

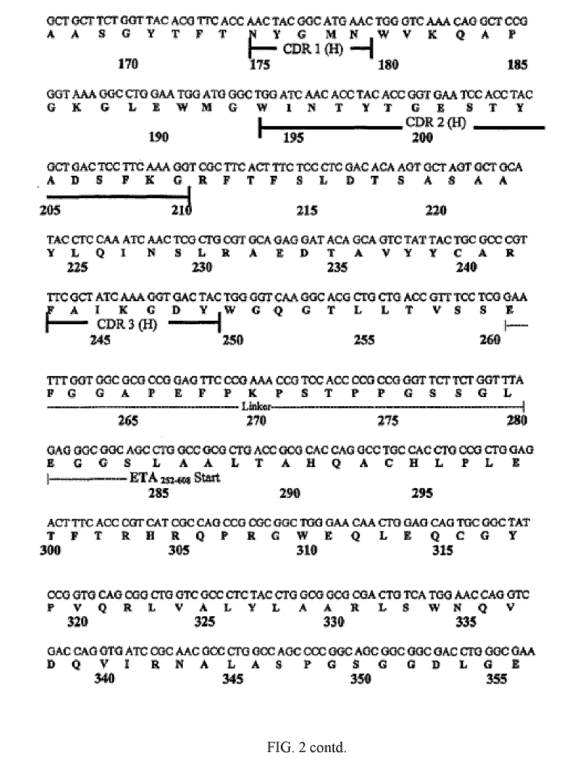

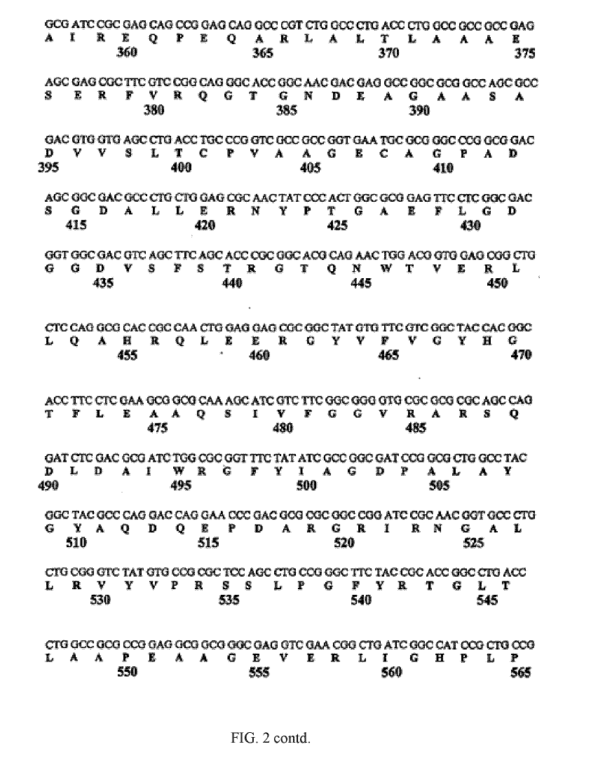

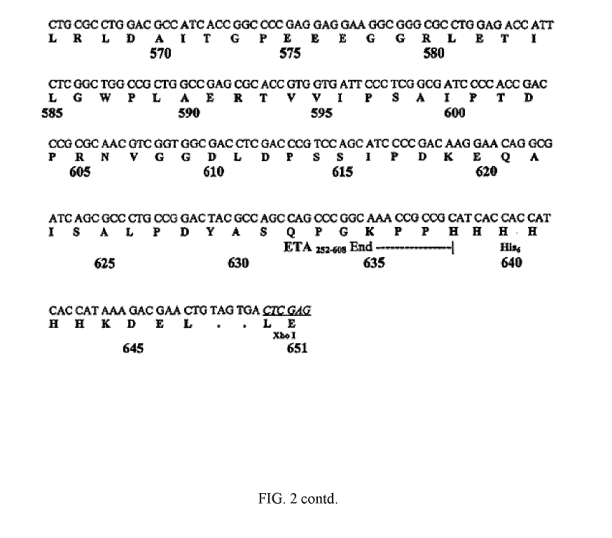

The gene sequence encoding a truncated form of the ETA (ETA252-608), and the Ep-CAM-binding 4D5MOCB scFv sequence were used to construct VB4-845. The molecule contains both N- and C-terminal His6 tails for purification, as depicted in FIG. 1. The DNA and amino acid sequence of VB4-845 are depicted in FIG. 2 and SEQ ID NOS: 3 and 2. The Ep-CAM binding portion is shown in FIG. 2. The CDR sequences are shown in SEQ ID NOS:4-9.

The resulting protein retains the specificity of the parent 4D5MOCB for Ep-CAM. The expression vector for the protein, pING3302 (Plasmid pING3302 from Xoma Ireland Ltd was used for the construction of the expression vector) is carried and expressed by the E 104 E. coli host strain. The protein is 648 amino acids in length and has a predicted molecular weight of 69.7 kilodaltons (kDa). In SDS-PAGE (sodium dodesyl sulfate-polyacrylamide gel electro-phoresis) analysis, VB4-845 is observed as a single protein band of approximately 70 kDa. The protein has an isoelectric point (pI) of approximately 5.9, and is water-soluble forming a clear solution.

### Example 2

### Dosage and Formulations

VB4-845 has been studied as a nascent drug and has been found to be effective in binding to tumor cell lines and in some model systems, preventing tumor growth. VB4-845 is formulated at 1 mg/ml in 20 mM sodium phosphate, 500 mM NaCl, pH 7.2, and can be administered by an intratumoral route with a 22-gauge needle. It is packaged in 1 ml borosilicate glass vials, closed with a gray butyl stopper and an aluminum overseal. Two fill sizes are currently available: 0.1 and 0.2 mL (0.1 mg and 0.2 mg VB4-845, respectively). Drug is stored at −70° C. The final product is not preserved and is for single use only.

### Example 3

### Stability of VB4-845

The sample product is labeled, stored, and shipped according to written and approved standard operating procedures. The product may be shipped under frozen conditions (e.g., on dry ice), and may be maintained, for example, at the study site in a limited access, controlled −70° C. freezer that is monitored regularly for temperature. The product may be maintained at this condition until time of use.

The shelf-life of the product when stored at −70° C. is at least six months. At physiological conditions (e.g., incubation of the drug product for four hours at 37° C. in PBS), the majority of the immunoconjugate molecules (at least 91%) are still eluted as monomers of the appropriate molecular weight (approximately 70 kDa). The amount of VB4-845 slowly decreases with time with no less than approximately 47% of the initial protein being present in monomeric form after twenty hours at 37° C. Similar results were obtained upon incubation of 99mTc-labeled VB4-845 in human serum, further corroborating the suitability of the immunoconjugate for in vivo application.

Short term stability studies have been conducted to evaluate the inherent stability of the investigational product under routine handling at the clinical site. VB4-845 was evaluated in its standard formulation at room temperature and at 2-8° C. In addition, VB4-845 was prepared in injection buffer of phosphate-buffered saline with and without 800 mM urea and tested up to six hours at room temperature. The short term stability studies also evaluated the impact of repeated freeze-thaw cycles on VB4-845.

VB4-845 was found to retain its biological activity over the course of all the short-term stability studies. VB4-845 may be withdrawn from the −70° C. freezer the day of dosing and allowed to thaw at room temperature. VB4-845 may be prepared into the injection buffer in 4-6 hours of its removal from the −70° C. storage condition. Once the product is formulated into the injection buffer of phosphate-buffered saline, the product may be injected into the patient within six hours of preparation. If the product cannot be used within a suitable time course, a new vial may be obtained from the inventory for dosing.

VB4-845 is stable in its original packaging for at least 20 hours at room temperature, and if kept refrigerated (e.g., at 2-8° C.), for at least 24 hours. If the product is unused, it can be refrozen for later use, particularly if the original container/closure system remains intact.

Short term stability studies (up to 16 hrs incubation time) in biogical fluid including human plasma, serum and urine demonstrated that VB4-845 retains it binding property and cell toxicity at least 16 hrs.

### Example 4

### Biodistribution

In general, the literature indicates that scFv are cleared rapidly from the circulation, and give high tumor-to-background ratios (specific retention in tumor mass) at early time points in animal models. T1/2 on average are 2-4 hours, but can be longer (>8 hours) depending upon the construction of the molecule and the route of administration. The highest uptake, depending on the molecule, tends to occur in the kidneys and liver after systemic infusion.

The biodistribution of VB4-845 has been assessed in mice bearing established Ep-CAM-positive SW2 and Ep-CAM-negative COLO320 xenografts at the contralateral flanks. The maximum dose of radiolabeled VB4-845 detected in SW2 tumors was 2.93% ID/g after four hours, which then gradually decreased to 1.95% ID/g and 1.13% ID/g after at 24 and 48 hours, respectively. In contrast, VB4-845 in COLO320 control tumors localized with a maximum dose of 1.65% ID/g after thirty minutes, which then rapidly declined to 1.06% ID/g after four hours and showed only background levels after 48 hours.

VB4-845 showed a, slower blood clearance than the parental scFv. After 24 hours, the total dose of VB4-845 in the blood was 0.42% ID/g, which was 1.5-fold more than the parent scFv (0.28% ID/g). Moreover, localization of the immunoconjugate in SW2 tumors was also delayed compared to the parent scFv, and the distribution of VB4-845 revealed a tumor:blood ratio of 5.38 after 48 hours, which was comparable to the ratio obtained with the scFv after 24 hours. At each time point, VB4-845 preferentially accumulated in Ep-CAM-positive SW2 tumors compared to COLO320 control tumor with a SW2:COLO320 ratio varying between 1.28 and 2.95. This indicates that VB4-845 was retained in Ep-CAM-positive tumors by specific antibody-antigen interactions and cellular uptake. The marginal accumulation in COLO320 control tumors may be due to the increase in vascular permeability often found in tumors. Analysis of normal tissues in these animals revealed that VB4-845 also localized in the kidney, spleen, liver and to a lower extent in the bone.

Clinical observations made during the conduct of the pharmacokinetic and efficacy models in mice indicate that the product was well tolerated without any clinical signs indicative of toxicity. All animals lived throughout the course of the studies and there was no drug related mortality.

### Example 5

### Preparation of VB4-845

Construction of the VB4-845 (also referred to as 4D5MOCB-ETA) expression vector. The sequence encoding a truncated form of ETA (ETA252-608) was amplified by PCR from plasmid pSW200 and cloned as an 1164 bp EcoRI-HindIII fragment downstream of the Ep-CAM-binding 4D5MOCB scFv sequence present in the pIG6-based 4D5MOCB scFv expression vector. The primers (Tox1: CTCGGAATTCGGTGGCGCGC CGGAGTTCCCGAAACCGTCCACCCCGCCGGGTTCTTCTGGTTTA (SEQ ID NO:10); Tox2: GTCAAGCTTCTACAGTTCGTCTTTATGGTGATGGTGGTGATGCGGCGGTTT CCCGGGCTG (SEQ ID NO:11) introduced an EcoRI restriction site between scFv and toxin and a C-terminal hexa-histidine tag followed by the endoplasmic reticulum (ER) retention signal KDEL, a stop codon and a HindIII restriction site. To improve purity and yield during IMAC, a second hexahistidine tag was added at the N-terminus between the periplasmic signal sequence and the 4D5MOCB coding region. To this end, two pairs of oligonucleotides (Xbal 5′: CTAGATAACGAGGGCAAAAAATGAAAAAGACAGCTATCGCGATTGCAGT GGCACTGGCTG-GTTTCGCTACCGT (SEQ ID NO:12); Xbal 3′: GCCACTGCAATCG CGATAGCTGTCTTTTTCATTTTTTGCCCTCGTTAT (SEQ ID NO:13); and EcoRV 5′: AGCGCAGGCCGACCACCATCATCACCATCACGAT (SEQ ID NO:14); EcoRV 3′: ATCGTGATGGTGATGATGGTGGTCGGCCTGCGCTACGGTAGCGAAACCAG CCAGT (SEQ ID NO:15) were heated to 80° C., allowed to anneal by gradually cooling to room temperature and then ligated between the Xbal and EcoRV sites of pIG6-4D5MOC BETAH6KDEL. The sequence was experimentally confirmed.

For periplasmic expression of VB4-845, the vector pIG6 was used, which places the gene under lac promoter control in SB536, an E. coli strain devoid of the periplasmic proteases HhoA and HhoB. Five ml 2YT medium containing ampicillin (100 mg/mL) were inoculated with a single bacterial colony containing the VB4-845 (4D5MOCB-ETA) expression plasmid and grown overnight at 25° C. The bacteria were diluted in one liter of 2YT medium supplemented with 0.5% glucose and ampicillin (100 mg/mL) to reach an A55O nm between 0.1 and 0.2 and transferred to 3-liter baffled shake flasks. The culture was further grown at 25° C. to an A55O nm of 0.5 and immunoconjugate production was induced for 4 h by adding a final concentration of 1 mM isopropyl-b-D-thiogalactopyranoside (IPTG, Sigma). The harvested pellet derived from a bacterial culture with a final A55O nm of 6 was stored at −80° C.

For purification, the pellet obtained from a one liter culture was re-suspended in 25 ml lysis buffer, containing 50 mM Tris-HCl (pH 7.5), 300 mM NaCl, 2 mM MgSO₄ and supplemented with EDTA-free protease inhibitor cocktail (Roche Diagnostics, Mannheim, Germany) and DNase I. The bacterial suspension was lysed with two cycles in a French Pressure Cell press (SLS Instruments, Urbana), centrifuged at 48,000 g in a SS-34 rotor for 30 min at 4° C. and subsequently filter-sterilized. The immunoconjugate present in the cleared supernatant was purified by chroma-tography using a BIOCAD-System (Perseptive BioSystems) with a Ni2+-iminodiacefic (IDA) column and a HQ/M-anion-exchange column coupled in-line as described. Before the lysate was loaded, the Ni²⁺-IDA column was equilibrated with 20 mM Tris (pH 7.5), 300 mM NaCl. After loading, the column was washed three times with different salt solutions, all buffered with 20 mM Tris (pH 7.5), in the order 300 mM, 510 mM and 90 mM NaCl. Subsequently, the column was washed with 20 mM Tris (pH 7.5), 10 mM imidazole, 90 mM NaCl, before the bound immunoconjugate was eluted with the same solution containing 200 mM imidazole (pH 7.5).

The eluate was directly loaded onto the HQ/M-an-ion-exchange column and the bound immunoconjugate was eluted with a salt gradient of 90-1000 mM NaCl, buffered with 20 mM Tris (pH 7.5). The fractions containing 4D5MOCB-ETA were collected and concentrated using a 10 kDa cutoff filter by centrifugation at 2000 g and 4° C. (Ultrafree-MC low protein binding, Millipore). The quality of purified VB4-845 (4D5MOCB-ETA) was analyzed by a 10% SDS-polyacrylamide gel and Western blotting using a horseradish peroxidase (HRP)-conjugated anti-tetrahistidine antibody (QIAGEN, Hilden, Germany) diluted 1:5000 according to the manufacturer's recommendations.

Analytical gel filtration and determination of thermal stability. Ten micrograms of purified VB4-845 (4D5MOCB-ETA) were diluted in 50 ml PBS pH 7.4 containing 0.005% Tween-20 and subsequently incubated at 37° C. Samples were analyzed at different time points (after 0 h, 2 h, 4 h, 8 h, 10 h and 20 h) by gel filtration using the Smart system (Pharmacia, Uppsala) with a Superose-12 PC3.2/30 column. The column was calibrated in the same buffer with three protein standards: alcohol dehydrogenase (Mr 150,000), bovine serum albumin (Mr 66,000) and carbonic anhydrase (Mr 29,000). The same analytical setting was used to assess the thermal stability of the 99m Tc-labeled immunoconjugate after a 20 h incubation at 37° C. in human serum. The amount of immunoconjugate monomers was determined by g-scintillation counting of the eluted fractions.

Radiolabeling and determination of antigen-binding affinity. VB4-845 (4D5MOCB-ETA) was radioactively labeled by stable site-specific coordination of 99mTc-tricarbonyl trihydrate to the hexahistidine tags present in the protein sequence. This spontaneous reaction was induced by mixing 30 ml of immunoconjugate solution (1 mg/ml) with one third volume of 1 M 2[N-morpholino]ethanesulfonic acid (MES) pH 6.8 and one third volume of freshly synthesized 99mTc-tricarbonyl compound. The mixture was incubated for 1 h at 37° C. and the reaction was stopped by desalting over a Biospin-6 column (BioRad, Hercules, Calif.) equilibrated with PBS containing 0.005% Tween-20, according to the manufacturer's recommendation. The percentage of immunoreactive immunoconjugate was assessed as described. The binding affinity of the 99mTc-labeled immunoconjugate was determined on SW2 cells in a radio-immunoassay (RIA), essentially as described for the scFv 4D5MOCB.

### Example 6

### Construction and Expression of VB4-845 with Codon-Optimized DNA Sequences

The expression yield of VB4-845 in E. coli was improved by modifying the coding and non-coding nucleic acid sequence of the expression vector. More specifically, the modifications included removing major pauses in the open reading frame, and details of which are disclosed in U.S. Pat. No. 8,318,472 which is incorporated herein by reference in its enterity. The codon-optimized DNA sequence encoding VB4-845 is represented by SEQ ID NO: 16. The codon-optimized VB4-845 DNA sequence was ligated into the pING3302 plasmid, and expressed in E. coli as in Example 5.

### Example 7

### Manufacturing Process

VB4-845 E. coli Fermentation. The production of VB4-845 is carried out in 2 L shake flasks using a rotary incubator shaker in a research laboratory. The rotary shaker resides within an environmental control room where temperature can be regulated to within one degree Celsius. Inoculation of seed medium, production medium and all aseptic manipulations take place under a biological safety cabinet type II/B with HEPA filtration and air classification of 100. Cell separation, concentration and diafiltration take place in a research laboratory.

VB4-845 is produced from the VB4-845 E104 host cell E. coli Master Cell Bank (MCB) (Plasmid pING3302 from Xoma Ireland Ltd was used for the construction of the expression vector). Initial scale-up of cell (fermentation) propagation for the production of clinical grade VB4-845 has been to the level of 26×2 L shake flasks with a working volume of 1 L per flask, total volume is 26 L. The VB4-845 E. coli MCB is grown in a complex nitrogen media containing glycerol as the principal carbon sources for cell growth. The fermentation procedure is described below.

Inoculum Preparation. For a 26 L shake flask run, one 500 mL culture of VB4-845 E. coli MCB is prepared as pre-inoculum. For each culture, a vial of MCB is withdrawn from the −18° C. storage tank and allowed to thaw at room temperature. The vial is wiped externally with 70% ethanol and allowed to air dry in a biological safety cabinet. The cell suspension of MCB (1.5 ml) is added to a 2 L Erlenmeyer flask containing 500 mL of sterile seed medium (modified 2YT medium and 25 mg/L tetracycline). The flask is transferred to a rotary shaker set at 200 rpm and grown at 25±1° C. until an optical density of 3.0±0.2 or greater is reached (10.5±1 hr, mid-log phase of growth). The inoculum is then used as a seed culture to inoculate the 26 production shake flasks.

Fermentation in 26×2 L shake flasks. Fermentation is carried out in 2 L-unbaffled flasks each containing 1 L of production medium. A typical production run for clinical grade VB4-845 has been 26×2 L flasks containing 1 L of production media (modified Terrific Broth, TB) per flask. The fermentation media is seeded with a 1% inoculum from the above culture and incubated on a shaker (200 rpm) at 25±1° C. until an optical density of 1.2 is reached (approximately 6-7 hours) at the last shake flask inoculated. A typical OD600 range at induction is 1.2-1.5. The VB4-845 expression is induced by the addition of 0.1% L-arabinose. Cells are harvested approximately 6 hours post-induction.

Cell Separation. At harvest, all shake flasks are removed from the shaker room in the order of inoculation, with the first inoculated flask removed first. The content of the first shake flask is added to the second shake flask under a biological hood. All subsequent shake flasks are removed likewise. The pooled shake flasks are placed in refrigeration at 2-8° C. The VB4-845 E104 E. coli cells are removed in groups of 6 from the above fermentation cultures by centrifugation at 6,800 g force for 15 minutes at 2-8° C. in a Sorvall and Beckman centrifuges. The cells are discarded while the cell free broth is retained for further processing. The concentrated cell suspension is collected, inactivated and disposed of by established methods. The resulting supernatant is pooled and a 5 ml sample is reserved for product quantification. The centrifuges, rotors and centrifuge bottles are thoroughly cleaned prior to processing the fermentation broth.

Concentration/Diafiltration. Concentration and diafiltration of harvested culture supernatant is performed by using a tangential flow Pellicon system with a Sartorius membrane (Hydrosart) molecular cut-off of 10 kD NMW (nominal molecular weight), and having a surface area of 3 square feet. The Pellicon filtration system is thoroughly washed prior to usage. Concentration is performed at a feed rate of 4 L/min and a permeate rate of 500 mL/min. A 5 ml sample is taken at the final concentration step. Diafiltration is performed against 0.02 M sodium phosphate, pH 7.2±0.2. Five volume changes are required to achieve the desired conductivity of <10 mS. The diafiltered concentrated product is clarified in a Sorvall centrifuge at 6,800 g force for approximately 30 minutes at a set temperature of 2-8° C. The clear solution-containing product of interest is filtered prior to purification using a 0.22 μm filter. The clarification step comprises, after diafiltration, centrifugation, passage through 0.2 μm Filter, addition of Triton X-100, adjustment of conductivity, adjustment of pH, and then follows purification.

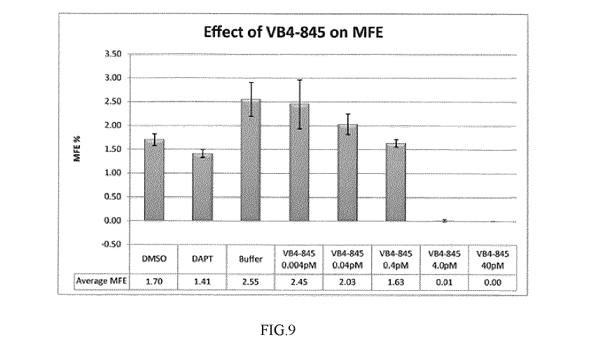

VB4-845 Purification Procedures. Purification of VB4-845 is performed in a cGMP controlled area with HEPA filtration and controlled environmental with air Classification of 10,000. The VB4-protein is isolated by metal-affinity chelating chromatography and is further purified by an anion exchange chromatography elution. The purification process is summarized in the flow diagram in FIG. 9, and is described below.

Chelating Sepharose Metal Interaction Chromatography. The metal-affinity column is prepared by packing chelating sepharose HP resin in a XK26/20 glass column, with a column volume of approximately 17±1 mL. The packing is performed at a backpressure of 3 bar. The working linear flow rate (LFR) is 90 cm/h. Five column volumes (CV) of water for injection (WFI) is passed through the chelating sepharose column. To charge the chelating sepharose column with metal ions, 5 CV of 0.1M nickel chloride solution is passed through the column. The remainder of the unbound nickel chloride is washed away with 5 CV of WFI. The column is then equilibrated with 10 CV of 20 mM sodium phosphate containing 150 mM sodium chloride and 0.1% Triton X-100, pH 7.2±0.1 buffer (chelating sepharose equilibration buffer).

The conductivity of the concentrated/diafiltered solution containing VB4-845 has been adjusted to 15±1 mS with sodium chloride and the pH is adjusted to 7.2±0.1 with 1M sodium hydroxide (NaOH). The VB4-845 containing solution is applied to the chelating sepharose HP column at a LFR of 90 cm/Hr or 8 ml/min. The column then is washed with 20 CV of wash buffer, 20 mM sodium phosphate, 150 mM sodium chloride, pH 7.2±0.1 buffer containing 20 mM imidazole and 0.1% Triton X-100 (wash buffer). The VB4-845 is eluted from the column with six CV of 20 mM sodium phosphate, 150 mM sodium chloride, pH 7.2±0.1 buffer, containing 500 mM imidazole (chelating sepharose elution buffer). The product is collected in a 3 CV fraction starting from the beginning of the elution peak.

Q-Sepharose—Anion Exchange Chromatography. The Q-Sepharose HP resin is packed in a XK16/20 glass column with a final column volume of 5.0±0.5 mL. The operating linear flow rate is 156 cm/h. The column is washed with 10 CV of WFI, then washed with 5 CV of 1M sodium chloride in 20 mM sodium phosphate, pH 7.2±0.1 buffer and equilibrated with 10 CV 20 mM sodium phosphate, 90 mM sodium chloride, pH 7.2±0.1 buffer (2-sepharose equilibration buffer). The elution from the chelating sepharose column is diluted with 20 mM sodium phosphate, pH 7.2±0.1 buffer until a conductivity of 10±1 mS is achieved. The partially purified VB4-845 is loaded onto the Q-Sepharose column at a flow rate of 5.2 ml/min to further reduce endotoxin levels and DNA. Once the product has been bound, the anion exchange column is washed with 15 CV of Q-Sepharose equilibration buffer. The contaminants are found in the flow-through and wash steps. The product is eluted with 20 mM sodium phosphate, 500 mM sodium chloride, pH 7.2±0.1 buffer as a 3 mL fraction.

### Example 8

### VB4-845 for Treatment of Hepatocellular Carcinoma (HCC) and for Killing Cancer Stem Cells

### Subjects and Tissue Samples

Between November 2008 and March 2010, 90 patients with HCC underwent curative hepatectomy at the Tokyo Medical and Dental University Hospital. The experimental methods used in determining the gross morphology type and analyzing the expression of Ep-CAM were performed as previously reported. Patients were followed up with assays of serum level of alpha-fetoprotein and protein induced by vitamin K absence or antagonists-II every month and with ultrasonography, computed tomography, and magnetic resonance imaging every 3 months. Median observation time was 25.2 months (95% confidence interval [CI], 10.4-37.7 months). Written informed consent was obtained from each subject, and study procedures were approved by the institutional review board.

### Cell Culture

Human HCC cell lines Hep3B, PLC/PRF/5, and SK-Hep1 were obtained from the American Type Culture Collection (Manassas, Va., USA). Other human HCC cell lines HuH-7, HuH-1, HepG2, HLE, and HLF were obtained from the Human Science Research Resources Bank (Osaka, Japan). HuH-7, HepG2, Hep3B, and SK-Hep1 cells were cultured in log growth phase in 1640 RPMI (Invitrogen, Carlsbad, Calif.) and HuH-1, HLE, HLF, and PLC/PRF/5, cells were cultured in Dulbecco's modified Eagle medium (Invitrogen) with 5% fetal bovine serum (Sigma, St. Louis, Mo.) for HLF cells or 10% FBS for the remaining cell lines. All media were supplemented with 1% PenStrep (Sigma). Luciferase expression plasmid pGL4.50[luc2/CMV/Hygro] vector (#E131A; Promega, Madison, Wis.) was transfected into HuH-7 cells according to the instructions of the manufacturer and luciferase-expressing HuH-7 cells (HuH-7-Luc) were generated. All cell lines were cultivated in a humidified incubator at 37° C. in 5% carbon dioxide and harvested with 0.05% trypsin-0.03% EDTA (Invitrogen).

### Flow Cytometry

For flow cytometry, FACSCanto™ II (BD Biosciences, San Jose, Calif.) was used. Cancer cells were washed with phosphate-buffered saline, and then enzymatically dissociated with 0.05% trypsin-EDTA (Invitrogen). The trypsinized cells were suspended in FACS buffer, and analyzed on FACSCanto™ II using FACSDiva software (BD Biosciences). For the analysis of hepatic stem/progenitor markers, primary antibodies against Ep-CAM (#324206; Biolegend, San Diego, Calif.), CD13 (#555394; BD Pharmingen), CD44 (#555479; BD Pharmingen), CD90 (#328110; BioLegend), CD133 (#130-080-801; Miltenyi Biotec, Gladbach, Germany), Mouse IgG1 κtype (#555749; BioLegend), and Mouse IgG2b κtype (#400314; BioLegend) were used. All antibodies were conjugated directly with phycoerythrin (PE). The immunostaining and analysis were performed according to the instructions from the manufacturer.

### Analysis of Cell Proliferation and Viability

HCC cell lines were seeded in 96-well plates at 3×10³ cells per well in a total volume of 50 μl of culture media. After 24 h, VB4-845 concentrations ranging from 0.001 to 10 pM were added in a total volume of 100 and further incubated for 72 h, or 5-FU concentrations ranging from 0.01 to 100 μg/ml were added and incubated for 48h under standard cell culture conditions. Cell viability was monitored using CellTiter 96 AQueous One Solution Cell Proliferation Assay Kit (Promega) and half-maximal inhibitory concentration (IC₅₀) values were calculated. The mean values and standard deviations of IC₅₀ were calculated in triplicate for each cell line. To investigate cell viability, HCC cell lines were seeded in 6-well plates at 1×10⁵ cells per well in a total volume of 2 ml of culture media. After 24 h, VB4-845 (1-10 pM) and 5-FU (1-5 μg/ml) were added and incubated for 48 h. The remaining viable cells were counted using a Cytorecon (GE Healthcare) after staining with trypan blue to exclude the dead cells. Each analysis was performed in triplicate, and the data expressed as means±standard deviations.

### Sphere Formation Assay

Briefly, 1×10⁶ cells of HuH-7, HepG2, Hep3B and HuH-1 were seeded in four 10 cm dishes. Twenty four hours later, PBS, VB4-845 (1-10 pM), 5-FU (1-5 μg/ml), and a combination of VB4-845 plus 5-FU were administered in each dish. After 48h, the medium was changed to drug-free medium and incubated for 24 h. After cell viability was confirmed by trypan blue exclusion, the remaining viable cells were collected and plated separately at 1×10² cells in low attachment plates (96-well Ultra Low Cluster Plate; Costar, Corning, N.Y.), and incubated in serum-free Dulbecco's modified Eagle medium/F12 medium (Invitrogen). Sphere formation was observed using AxioObserver (Carl Zeiss, Oberkochen, Germany), and the images were acquired digitally using AxioVision software (Carl Zeiss).

### Immunohistochemical Analysis

Immunohistochemical analysis of Ep-CAM was performed on tissue sections of tumors using an anti-Ep-CAM antibody (# ab71916; Abcam, Cambridge, UK) at 1:160 dilutions with PBS, followed by reactions in an automated immunostainer (Ventana; Tucson, Ariz., USA) using heat-induced epitope retrieval and a standard DAB detection kit (Ventana). The tumor cells showed equivalent membranous staining to normal bile duct epithelium that was defined as strongly-stained tumor cells. The immunostaining was evaluated quantitatively by counting in no fewer than 3 different random fields (100× magnification) under a light microscope by two independent investigators. The data are expressed as means±standard deviations.

### In Vivo Studies in a Subcutaneous Xenograft Model

A subcutaneous tumor model was used to analyze the in vivo activity of VB4-845. Twenty five-week-old female NOD.CB17-PRkdcScid/J mice purchased from Charles River Laboratory Inc. (Kanagawa, Japan) were injected with 1×10⁶ HuH-7 cells mixed with the same amount of Matrigel (BD Biosciences) subcutaneously into the both flanks of mice under anesthesia. Palpable tumors were confirmed in all 40 injection sites two weeks after the inoculation, and mice were randomized into four groups (n=5): control, VB4-845 (30 μg/kg), 5-FU (30 mg/kg), and a combination of VB4-845 and 5-FU. Saline or VB4-845 diluted in 100 μl of saline was injected by tail vein injection, and saline or 5-FU diluted in 100 μl of saline was injected intraperitoneally three times a week for 2 weeks for a total of 6 doses. Tumor size was measured using calipers three times a week and tumor volumes calculated using the following equation: volume=(length)×(width)2×0.5. The mice were sacrificed three weeks after the initiation of treatment.

### In Vivo Studies in a Orthotopic Liver Xenograft Model

An orthotopic xenograft model was created by direct intrahepatic inoculation of HuH-7-Luc cells. Ten five-week-old female NOD.CB17-PRkdc^{scid/}J mice were fully anesthetized and 5×10⁵ cells suspended in 20 μl of Matrigel (BD Biosciences) were slowly injected into the upper left lobe of the liver. Three weeks after the inoculation, the luciferase-luciferin-based imaging using IVIS system (Xenogen, Alameda, Calif.) was used for monitoring the correct implantation in the liver. All mice exhibited liver tumors and were randomized into two groups; control group and the combination of VB4-845 and 5-FU group (5 mice in each). The method of administration was the same as the subcutaneous model. Two weeks after the initiation of treatment, mice were sacrificed and the size of liver tumor was measured.

### Results

### Prospective Studies of Ep-CAM Expression in Confluent Multinodular (CM)-Type HCC and the Patient Prognosis

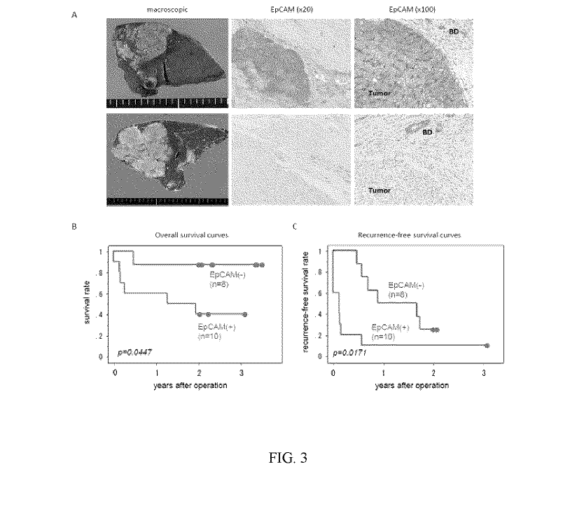

Among 90 patients with HCC, 18 cases were diagnosed as CM-type, “a unifocal but multinodular and well-demarcated tumor, without any identifiable large tumor nodule suggesting a primary focus”, according to the General Rules for the Clinical and Pathological Study of Primary Liver Cancer by Liver Cancer Study Group of Japan. As shown in FIG. 3A, Ep-CAM expression in HCC cells was observed in 10 cases, but not in the remaining 8 cases. The prognostic significance of Ep-CAM expression was then prospectively evaluated. It is noteworthy that a significant relationship was observed between Ep-CAM expression and the patient prognosis (FIG. 3B; p=0.0447) as well as recurrence (FIG. 3C; p=0.0171).

### Expression of Ep-CAM in Human HCC Cells

The expression of Ep-CAM was assessed using FACS analysis in 8 human HCC cell lines, as shown in FIG. 4A. These cell lines were divided into two groups: Ep-CAM-high-expression (Ep-CAM^{high}) HCC cell lines including HuH-7 (98.0±0.3%), HepG2 (98.0±0.9%), Hep3B (99.8±0.1%), and HuH-1 (97.7±0.2%) in which more than 95% of cells were positive for Ep-CAM and Ep-CAM-low-expression (Ep-CAM^{low}) HCC cell lines including HLE (0.4±0.1%), HLF (0.4±0.2%), PLC/PRF/5 (4.0±0.3%), and SK-Hep1 (0.7±0.2%) in which less than 5% of cells were positive for Ep-CAM. There was no difference in proliferation activity and morphological features between these two groups.

### In Vitro Effects of VB4-845 Plus 5-FU in Human HCC Cells

The effect of VB4-845 was analyzed in human HCC cell lines. VB4-845 was effective for Ep-CAM^{high} cell lines but not for the Ep-CAM^{low} cell lines, as shown in FIG. 4B. The IC₅₀ value of VB4-845 was 4.6±0.1×10⁻² pM for HuH-7, 1.0±0.1×10⁻² pM for HepG2, 0.9±0.1×10⁻² pM for Hep3B, and 7.3±0.2×10⁻² pM for HuH-1. On the other hand, VB4-845 had no effect against Ep-CAM^{low} cell lines at all and was unable to determine IC₅₀ values with these cell lines. As shown in FIG. 4C, 5-FU showed potent anti-proliferative activity in all HCC cell types with IC₅₀ value of 0.8±0.1 μg/ml for HuH-7, 39.5±9.6 μg/ml for HepG2, 5.9±1.8 μg/ml for Hep3B, 11.3±6.3 μg/ml for HuH-1, 16.5±6.6 μg/ml for HLE, 33.5±17.2 μg/ml for HLF, 55.6±11.2 μg/ml for PLC/PRF/5, 4.3±0.5 μg/ml for SK-Hep1 cells. There was no significant correlation between the efficacy of 5-FU and the expression of Ep-CAM in each cell line (R=0.16, p=0.38).

The combination effects of VB4-845 and 5-FU were assessed in 8 human HCC cell lines, as shown in FIG. 4D. The combination of VB4-845 and 5-FU significantly suppressed cell proliferation in all of the Ep-CAM^{high} cell lines (p<0.05). However, in the Ep-CAM^{low} cell lines, 5-FU suppressed cell proliferation to the same extent as the combination of VB4-845 and 5-FU (p>0.05), thus these cell lines did not demonstrate the combined effects of both drugs. Therefore, the Ep-CAM^{high} cell lines were chosen for further analysis.

Sphere Formation Assay after the Treatment of VB4-845, 5-FU, and the Combination of VB4-845 Plus 5-FU

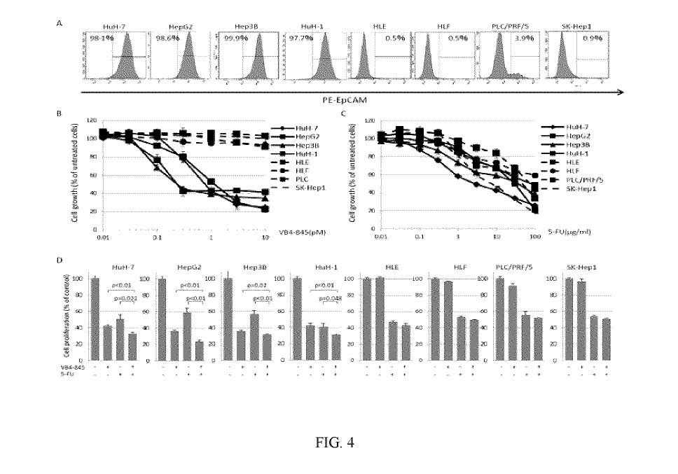

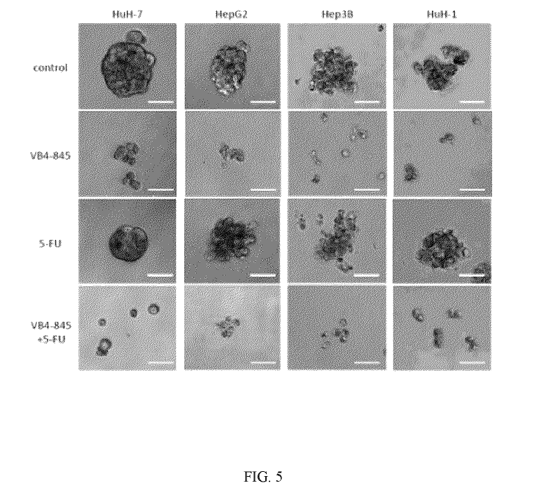

After the treatment with VB4-845, 5-FU, and the combination of VB4-845 plus 5-FU on Ep-CAM^{high} cell lines, the viable cells were collected and analyzed for their ability to form spheres on re-culturing (FIG. 5). The surviving cells of the 5-FU treatment formed spheres in all of the 4 cell lines after 7 days of culture, whereas the surviving cells remaining after exposure to VB4-845 alone or in combination with 5-FU did not form such spheres after 7 days of culture (FIG. 5). Although the doses of VB4-845 and 5-FU used in this assay showed similar anti-proliferative activity, their effects on sphere forming ability were in direct opposition to one another. Since the sphere-forming cells are assumed to be capable of self-renewal, one of essential hallmarks of stemness, the effect of VB4-845 for Ep-CAM^{high} cell lines was found to be closely associated with their stemness.

Alterations of Stem/Progenitor Markers after the Treatment of VB4-845, 5-FU, and the Combination of VB4-845 Plus 5-FU

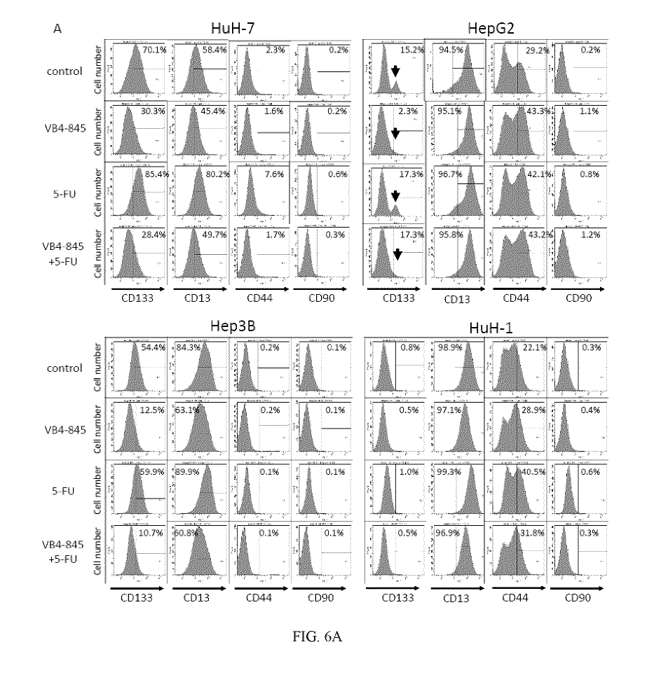

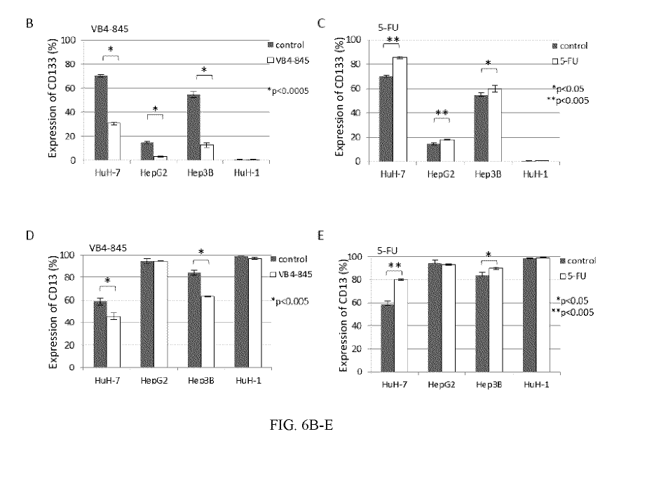

In the Ep-CAM^{high} cell lines, the expression of several stem/progenitor markers such as CD133, CD13, CD44, and CD90 was analyzed using FACS analysis after the treatment of VB4-845, 5-FU, and the combination of VB4-845 plus 5-FU, as shown in FIG. 6A. These markers were chosen because they were reported as biomarkers of HCC with poor prognosis. The positive rate of CD133 in HuH-7, HepG2, and Hep3B cells was significantly decreased after the administration of VB4-845 compared with the control, as shown in FIG. 6B (p<0.0005). Interestingly, HepG2 cells showed a unique bimodal pattern for CD133 expression (FIG. 6A, arrow) and the administration of VB4-845 dramatically decreased this CD133-positive subpopulation of HepG2 cells with statistical significance. On the other hand, the administration of 5-FU significantly increased the positive rate of CD133 in HuH-7, HepG2, and Hep3B cells compared with the control, as shown in FIG. 6C (p<0.05). The positive rate of CD13 in HuH-7 and Hep3B cells was significantly decreased with the treatment of VB4-845 (FIG. 6D, p<0.01) and significantly increased with the treatment of 5-FU (FIG. 6E, p<0.05). There was no consistent tendency for the positive rate of CD44 and CD90 after the treatment of each cell line. These results indicate the effect of VB4-845 might be closely associated with the stemness of human HCC cells.

### In Vivo Effects of VB4-845, 5-FU, and the Combination of VB4-845 Plus 5-FU

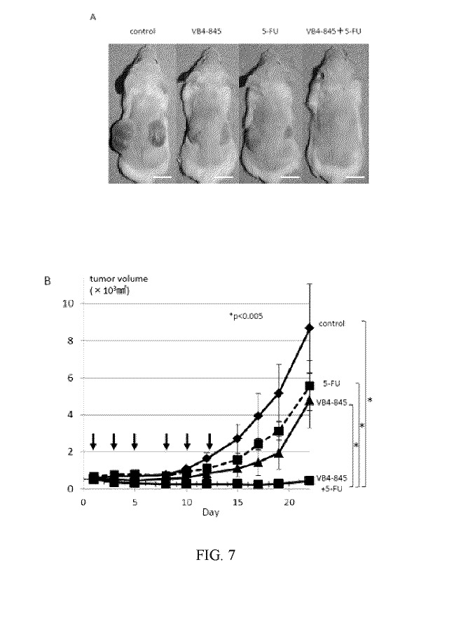

To investigate in vivo antitumor activity, NOD.CB17-PRkdc^{Scid/}J mice bearing established HuH-7 subcutaneous xenografts were utilized. Three weeks after the initiation of treatment, mice were sacrificed and the volume of tumors was measured (865±238 mm³ in the control group, 476±134 mm³ in the VB4-845 treated group, 555±147 mm³ in the 5-FU treated group, and 43±8.4 mm³ in the combination of VB4-845 plus 5-FU treated group). As shown in FIGS. 7A and 7B, the volume of the tumors in the VB4-845 and 5-FU monotherapy groups appeared smaller, when compared with the control group (p=0.078 and 0.31, respectively). Significant tumor regression was observed in the group treated with VB4-845 plus 5-FU compared with the control group, the VB4-845 treated group, and the 5-FU treated group (p<0.05). None of the treated mice showed signs of wasting or other toxicity relative to control mice. The data demonstrates that VB4-845 and 5-FU had different effects on the sphere-forming ability and on the populations expressing hepatic stem/progenitor markers. These different effects related to the stemness of tumor cells may be closely associated with the significant regression of the tumor in the combination group.

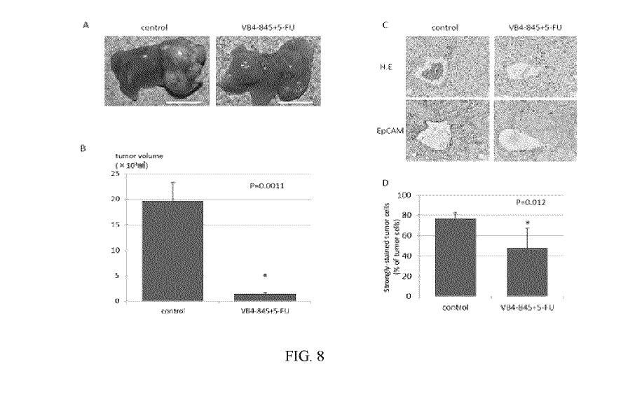

As shown in FIGS. 8A and 8B of the orthotopic xenograft model, the combined therapy of VB4-845 and 5-FU significantly suppressed the liver tumors in all mice (141±34 mm³) compared with the control (1964±367 mm³) (p=0.011). The immunohistological expression of Ep-CAM (FIG. 8C) demonstrated that the population of strongly-stained tumor cells was decreased in VB4-845 plus 5-FU group (47.4±19.4%) compared with the control group (76.7±6.0%) (FIG. 8D, p=0.012). None of the treated mice showed signs of wasting or other toxicity relative to control mice. All host tissues examined, including liver, bone marrow, kidney, intestine and lung, were histologically normal in all experiments.

### Discussion

In this study, FACS analysis of Ep-CAM expression revealed that 8 human HCC cell lines were classified into two groups; 4 Ep-CAM^{high} (>95%) and 4 Ep-CAM^{low} (<5%) HCC (FIG. 4A). Since the close correlation between Ep-CAM expression and sphere-formation was reported in HCC cells, the effect of VB4-845 on sphere forming ability was analyzed. Although 5-FU treatment did not affect the sphere formation, the treatment with VB4-845 as well as the combination of VB4-845 plus 5-FU clearly suppressed the sphere formation in all 4 HCC cell lines, shown in FIG. 5. Since the sphere-forming ability is known to be regulated by the self-renewing capacity of stem cells, the effects of VB4-845 might be closely associated with the sternness of Ep-CAM^{high} HCC cells.

For further investigation of the VB4-845 effects on the sternness, the expression of several stem/progenitor markers such as CD133, CD13, CD44, and CD90 was analyzed after the treatment of VB4-845, 5-FU, and the combination of VB4-845 plus 5-FU. As shown in FIG. 6C, 5-FU treatment significantly increased the positive rates of CD133 in 3 HCC cell lines (p<0.05). Furthermore, VB4-845 dramatically decreased the CD133+ subpopulations in these HCC cells (FIG. 6B, p<0.0005). Similar results were obtained from the analysis of another stem cell marker CD13. The positive rates of CD13 were significantly decreased by VB4-845 treatment (FIG. 6D) but increased by 5-FU treatment (FIG. 6E). These results indicated that the targeted subpopulations were different between the VB4-845 and 5-FU treatments. With respect to the stem/progenitor markers, the effects of VB4-845 were also found to be closely associated with the sternness of human HCC cells.

Additionally, in vivo antitumor effects of VB4-845 and/or 5-FU were analyzed using the subcutaneous xenograft model as well as the liver orthotopic xenograft model. In the subcutaneous xenograft model (FIG. 7), antitumor effect was detected by either VB4-845 or 5-FU monotherapy, and the combination therapy of VB4-845 and 5-FU further decreased the tumor volume. Since the organ microenvironment in cancer might play a critical role in drug sensitivity, particularly for HCC, an organotropic cancer, a liver orthotopic xenograft model having similarity with the clinical condition was also utilized to explore tumor growth inhibition. As observed in the subcutaneous xenograft model, significant regression of tumors was observed in the VB4-845 plus 5-FU treated group compared with the control group (FIG. 8, p=0.0011).

Without wishing to be bound by theory, these studies show Ep-CAM-targeted therapy appears to demonstrate anti-cancer effects via potentially different mechanism (e.g., sternness) from the conventional cytotoxic agent 5-FU. Indeed, the preclinical studies show that the combination therapy of an immunoconjugate targeting for Ep-CAM with a conventional cytotoxic agent is a promising novel approach for the treatment of human HCC. Further studies and clinical trials of Ep-CAM-targeting agents will confirm its therapeutic role in the HCC management.

### Example 9

### Effect of VB4-845 on Beast Cancer Stem Cells

MCF-7 breast cancer cells were placed in a single cell suspension at low density and cultured for 7 days in serum-free media in poly-HEMA-coated 6-well plates to prevent cell adhesion. VB4-845 and controls were added into the culture medium at the time of plating (n=3 wells per concentration) at multiple concentrations. Vehicle controls and γ-secretase inhibitor DAPT (50 μM) were also included (FIG. 9). The non-stem-like cells die leaving the stem-like cells to persist and proliferate to form mammospheres. After 7 days, the number of mammospheres over 50 μm in size were counted per well and a mammosphere forming efficiency (MFE) calculated for the test agent and controls.

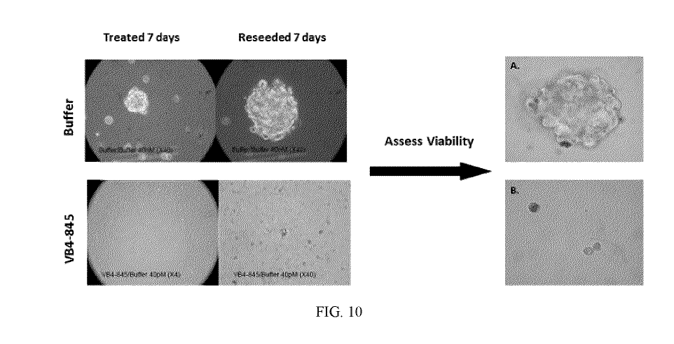

To assess whether the reduction in mammospheres was due to the killing of stem cells and not due to a block in proliferation, VB4-845-treated cultures were harvested and re-plated in 6-well poly-HEMA-coated plates for 7 days in the presence/absence of VB4-845 and cytotoxicity measured by Trypan Blue exclusion (FIG. 10). VB4-845 demonstrated the ability to completely inhibit sphere formation in the primary sphere assay. This effect was also observed at the concentrations tested in the re-plating assay whereby no spheres were formed when the test items were removed from the culture media. Cytotoxicity was confirmed with Trypan Blue added to the media of the cells on Day 10 post re-plating.

These studies demonstrated the ability of VB4-845 to completely inhibit sphere formation in the pM range. Re-plating of cells treated with VB4-845 failed to produce any spheres, indicating the cytotoxic effect.

1. An effective amount of an immunoconjugate for use in treating or preventing hepatocellular carcinoma at a cancer site, wherein said immunoconjugate comprises an antibody fragment conjugated to an effector molecule, and wherein the antibody binds epithelial cell adhesion molecule (Ep-CAM).

2. The immunoconjugate of claim 1, wherein the antibody fragment is murine, humanized, or a chimeric antibody.

3. The immunoconjugate of claim 1, wherein antibody fragment is selected from the group consisting of Fab, Fab′, F(ab′)2, scFv, dsFv, ds-scFv, dimers, minibodies, diabodies, bispecific antibody fragments, multimers, and any combination thereof.

4. The immunoconjugate of claim 1, wherein the antibody fragment comprises light chain complementarity determining regions comprising the amino acid sequences defined by SEQ ID NOS: 4, 5, and 6, and heavy chain complementarity determining regions comprising the amino acid sequences defined by SEQ ID NOS: 7, 8, and 9.

5. The immunoconjugate of claim 1, wherein the immunoconjugate comprises an amino acid sequence from amino acid 23 to amino acid 669 of SEQ ID NO: 2 (VB4-845).

6. A method of detecting or monitoring hepatocellular carcinoma in a subject comprising the steps of:
contacting a test sample taken from said subject with an antibody to form an antibody-antigen complex, wherein the antibody comprises light chain complementarity determining regions comprising the amino acid sequences defined by SEQ ID NOS: 4, 5 and 6, and heavy chain complementarity determining regions comprising the amino acid sequences defined by SEQ ID NOS: 7, 8, and 9;
measuring the amount of antibody-antigen complex in the test sample; and
normalizing the results against a control.

7. A kit for diagnosing hepatocellular carcinoma comprising:
an antigen comprising light chain complementarity determining regions comprising the amino acid sequences defined by SEQ ID NOS: 4, 5 and 6, and heavy chain complementarity determining regions comprising the amino acid sequences defined by SEQ ID NOS: 7, 8, and 9; and
instructions for the use thereof.

8. A method of killing liver cancer cells in vitro or in vivo comprising contacting the liver cancer cells to an effective amount of an immunoconjugate comprising an antibody conjugated to an effector molecule, and wherein the antibody recognizes epithelial cell adhesion molecule (Ep-CAM).

9. The method of claim 8, wherein the antibody comprises light chain complementarity determining regions comprising the amino acid sequences defined by SEQ ID NOS: 4, 5, and 6, and heavy chain complementarity determining regions comprising the amino acid sequences defined by SEQ ID NOS: 7, 8, and 9.

10. The method of claim 8, wherein the antibody comprises a polypeptide comprising heavy chain variable region and a light chain variable region as shown in SEQ ID NO: 1.

11. The method of claim 8, wherein the immunoconjugate comprises an amino acid sequence from amino acid 23 to amino acid 669 of SEQ ID NO: 2 (VB4-845).

12. The method of claim 8, further comprising contacting liver cancer cells with the immunoconjugate along with an anticancer agent.

13. The method of claim 12, wherein the anticancer agent is selected from tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, vincristine, taxol, taxotere, etoposide, teniposide, amsacrine, Irinotecan, topotecan, epothilones, gefitinib, erlotinib, angiogenesis inhibitors, EGF inhibitors, VEGF inhibitors, CDK inhibitors, cytokines, Her1 and Her2 inhibitors, and monoclonal antibodies.

14. A method of treating a subject with hepatocellular carcinoma comprising:
administering to the subject a therapeutically effective amount of an immunoconjugate comprising an antibody conjugated to an effector molecule, and wherein the antibody binds epithelial cell adhesion molecule (Ep-CAM).

15. The method of claim 14, wherein the antibody comprises light chain complementarity determining regions comprising the amino acid sequences defined by SEQ ID NOS: 4, 5, and 6, and heavy chain complementarity determining regions comprising the amino acid sequences defined by SEQ ID NOS: 7, 8, and 9.

16. The method of claim 14, wherein the antibody comprises a polypeptide comprising heavy chain variable region and a light chain variable region as shown in SEQ ID NO: 1.

17. The method of claim 14, wherein the antibody is an antibody fragment selected from the group consisting of Fab, Fab′, F(ab′)2, scFv, dsFv, ds-scFv, dimers, minibodies, diabodies, bispecific antibody fragments, multimers, and any combination thereof.

18. The method of claim 14, wherein the effector molecule is selected from the group consisting of radioisotopes, antineoplastic agents, immunomodulators, biological response modifiers, lectins, toxins, and any combination thereof.

19. The method of claim 14, wherein the effector molecule is a toxin selected from the group consisting of abrin, modeccin, viscumin, gelonin, bouganin, saporin, ricin, ricin A chain, bryodin, luffin, momordin, restrictocin, Pseudomonas exotoxin A, pertussis toxin, tetanus toxin, botulinum toxin, Shigella toxin, cholera toxin, diphtheria toxin and any combination thereof.

20. The method of claim 14, wherein the immunoconjugate is internalized by the cancer cell.

21. The method of claim 14, wherein the immunoconjugate comprises an amino acid sequence from amino acid 23 to amino acid 669 of SEQ ID NO: 2 (VB4-845).

22. The method according to claim 14, wherein the administration of the immunoconjugate is directly to the cancer site.

23. The method of claim 14, wherein the immunoconjugate is administered Sin combination with one or more anticancer agents.

24. The method of claim 14, wherein the immunoconjugate is administered in combination with 5-fluorouracil.

25. The method of claim 24, wherein the immunoconjugate is VB4-845 and is administered at a dosage of about 100 micrograms/day to about 2500 micrograms/day, and 5-fluorouracil is administered at a dosage of about 2 mg/kg/day to about 20 mg/kg/day.

26. The method of claim 23, wherein the immunoconjugate is co-administered, concurrently administered, or sequentially administered with one or more anticancer agents.

27. The method of claim 23, wherein the anticancer agent is selected from tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, vincristine, taxol, taxotere, etoposide, teniposide, amsacrine, Irinotecan, topotecan, epothilones, gefitinib, erlotinib, angiogenesis inhibitors, EGF inhibitors, VEGF inhibitors, CDK inhibitors, cytokines, Her1 and Her2 inhibitors, and monoclonal antibodies.

28. The method of claim 14, wherein the immunoconjugate is administered to the subject before the cancer treatment, concurrently with the cancer treatment, post-treatment, or during remission of the cancer.

29. The method of claim 14, wherein the immunoconjugate is VB-845 and is administered at a dosage of about 100 micrograms/day to about 2500 micrograms/day, for 7 to 21 days.

30. The method of claim 14, wherein the immunoconjugate is VB-845 and is administered at a dosage of about 500 micrograms/day to about 2500 micrograms/day, for 7 to 21 days.

31. The method of claim 14, wherein the immunoconjugate is VB-845 and is administered at a dosage of about 300 micrograms/day, for 7 to 21 days.

32. The method of claim 14, wherein the immunoconjugate is VB-845 and is administered at a dosage of about 500 micrograms/week to about 5000 micrograms/week, for 4 weeks.

33. The method of claim 14, wherein the immunoconjugate is VB-845 and is administered at a dosage of about 700 micrograms/week, for 4 weeks.

34. The method of claim 14, wherein the immunoconjugate is VB-845 and is administered at a dosage of about 1000 micrograms/week, for 4 weeks.
